SEQUENCE LISTING (28 sequences)

SEQ ID NO: 1 - protein, 225 aa, Homo sapiens
  wild type
AHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 225 aa, Homo sapiens
  110B09
AHTCPPCPAPELLGGPSVFLFPPKPKDTLMISRTPEVTCVVVDVSHEDPEVKFDWYVDGVEVHNAKTKPQEEQYNSTYRVVSALTVLHQDWLNGKEYKCKVSNKALPAPIEKTISKAKGQPREPQVYTLPPSREEMTKNQVSLTCLVKGFYPSDVAVEWESNGQPENNYKTAPPVLDSDGSFFLYSKLTADKSRWQQGNVFSCSVMHEALHNHYTQESLSLSPGK

SEQ ID NO: 3 - protein, 225 aa, Homo sapiens
  110H08
AHTCPPCPAPELLGGPSVFLFLPEPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLGSDGSFFLYSRLTVDKSRWQQGNVFSCSVMHEALHNHYTQKSLSLSPGK

SEQ ID NO: 4 - protein, 225 aa, Homo sapiens
  115A02
AHTCPPCPAPELLGGPSVFLFPPKPKDTLMISRTPEVTCVVVDVSHEDPEVKFNWYVDGVEVHNAKTKPREEQYNSTYRVVGVLTVLHQDWLNGKEYRCKVTNEALPAPIEKTISKAKGQPREPQVYTLPPSREETTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 225 aa, Homo sapiens
  121F02
AHTCPPCPAPELLGGPSVFLSPPKLKDTLMISRTPEVTCVVVVASHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWENNGQPENNYKTTPPVLDSDGSFFLYSKLIVDKSRWQQGNVFSCSVMHEALHNHYTQKSLSLSPGK

SEQ ID NO: 6 - protein, 225 aa, Homo sapiens
  125B01
ANTCPPCPAPELLGGPSVFLLPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKATPPVLDSDGSFFLYSKLTVDKSRWQQGNVFSCSVMHEALPNHYTQKSLSLSPGK

SEQ ID NO: 7 - protein, 225 aa, Homo sapiens
  125E05
AHTCPPCPAPELLGGPSAFLFPPKPKDTLMISRTPEVTCVVVDVGHEDPEVKFNWYVDGVEVHNAKTKPREEQYNSTYRVVSVLTVLHQDWLNGKEYKCKVSNKALPAPIEKTISKAKEQPREPQVYTLPPSRGEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 225 aa, Homo sapiens
  125H10
AHTCPPCPAPELLGGPSVFLFPPKPKDTLTISRTPEVTCVVVDVSHEDPEVKFNWYVDGVEVHNAKTKLREEQYNSTYRVVSVLTVLHQDWLNGKEYKCKVSNKALPAPIEKTISKAKGQPREPQVYTLAPSWEEMTKNQVSLTCLVKGFYPSDIAVEWESNGQPENDYKTTPPVLDSDGSFFLYGKLTVDKSRWQQGNVFSCFVMHESLHNHYTQKSLSLSPGK

SEQ ID NO: 9 - protein, 225 aa, Homo sapiens
  126F07
AHTCPLCPAPELLGGPSVFLFPPKPKDTLMISRTPEVTCVVVDVSHEDPEVKFNWYVDGVEVHNAKAKPREEQYNSTYRVVSVLTVLHQDWLNGKEYKCKVSNKALPAPIEKTISKAKGQPREPQVYTLPPSREEMTKNQVSQTCLVKGFYPSDIAVEWESNGQPESNYKTTPPVLDSDGSFFLYSKLTVDKSRWQQGNVFSCSVMHEALHNHYTQKGLSLSPGK

SEQ ID NO: 10 - protein, 225 aa, Homo sapiens
  126G05
AHTCPPCPAPELLGGPSVLLFPPKPKDTLMISRTPEVTCVVVDVSHEDPEAKFNWYVDGVEVHNAKTKPREEQYNSTYRVVSVLTVLHQDWLNGKEYKCKVSNKALPAPIEKTISKAQGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSFSLSPGK

SEQ ID NO: 11 - protein, 225 aa, Homo sapiens
  130A02
AHTCPPCPAPELLGGPSVLLFPPKPKDTLMISRTPEVTCVVVDVSHEDPEVKFNWYVDGVEVHNAKTKPREEQYNSAYRVVSVLTVLHQDWLNGKEYKCKVSNKALPAPTEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVTHEALHNHYTQKSLSLSPGK

SEQ ID NO: 12 - protein, 225 aa, Homo sapiens
  135A09
AHTCPPCPAPELLGGPSVFLFPPKPKDTLMISRTPEVTCVVVDVSHKDPEVKFNWYVDGVEVHNAKTKPREEQYNSTHRVVSVLTVLHQDWLNGKEYKCKVSNKALPAPIEKTISKAKGRPREPQVYTLPPSREEMTKNQVSLTCLVKGFYPSDIAVEWESNGQPENNYKTTPPVLDSDGSFFLYSKLTVDKSRWQQGNIFSCSVMHEALHNHYTQKSLSLSPAK

SEQ ID NO: 13 - protein, 225 aa, Homo sapiens
  135C09
AHACPPCPAPELLGGPSVFLFPPKPKDTLMISRTPEVTCVVVDVSHEDPEVKFNWYVDGVEVHNAKTKPREEQYNSTYCVVGVLTVLHQNWLDGKEYKCKVSNKALPAPIEKTISKAKGQPREPQVYTSPPSREEMTKNQVSLTCLVKGFYPSDIAVEWESNGQPENNYKTTPPVLDSDGSFFLYSKLTVDKSRWQQGSVFSCSVMHEALHNHYTQKSLSLSPGK

SEQ ID NO: 14 - protein, 225 aa, Homo sapiens
  135D10
AHTCPPCPAPELLGGPSVFLFPPKPKDTLMITRTPEVTCVVVDVSHEDPEVKFNWYVDGVEVHNAKTKPREEQYNSTYRVVSVITVLHQDWLNGKEYKCKVSNRALPAPIEKTISKAKGQPREPQVYTLPPSREEMTKNLVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 225 aa, Homo sapiens
  135E07
AYTCPPCSAPELLGGPSVFLFPPKPKDTLMISRTPEVTCVVVDVSHEDPEVKFNWYVDGVEVHNAKTKPREEQYNSTYRVVSVLTVLHQDWLNGKEYKCKASNKALPAPIDKTISRAKGQPREPQVYTLPPSREEMTKNQVCLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 225 aa, Homo sapiens
  136D04a
AHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKIISKAKGQPREPQVYTLPPSREEMTKNQVSLTCLVKGFYPSDIAVEWESNGQPENNYKTTPPVLDSDGSFFLYSKLTVDMSRWQQGNVFSCSVMHEALHNHYTQKSLSLSRGK

SEQ ID NO: 17 - protein, 225 aa, Homo sapiens
  136D04b
AHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKIISKAKGQPREPQVYTLPPSREEMTKNQVSLTCLVKGFYPSDIAVEWESNGQPENNYKTTPPVLDSDGSFFLYSKLTVDMSRWQQGNVFSCSVMHEALHNHYTQKSLSLSPGK

SEQ ID NO: 18 - protein, 225 aa, Homo sapiens
  136G11
AHTCPPCPAPELLGGPSVFLFPPKAKDTLMISRTPKVTCVVVDVSHEDPEVKFNWYVNGVEVHNARTKPREEQYDSAYRVVSVLTVLHQDWLNGKEYKCEVSNKALPAPIEKTISKAKGRPREPQVYTLPPAREEMTKNQVSPTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 225 aa, Homo sapiens
  136H08
AHTCPPCPAPELLGGPSVFLFPPKPKDTLMISRTPEVTCVVVDVSNEDPEVKFNWYMDGVEVHNAKTKPREEQYNSTYRVVSVLTVLHQDWLNGKEYKCKVSNKALPAPIEKTISKTKGQPREPQVYTLPPSREEMTKDQVSLTCLVKGFYPSDIAVEWESNGQPENNYKTTPPVLDSDGSFFLYSKLTVDKSRWQQGNVFSCPVMHEALHNHYTQKSLSLSPGK

SEQ ID NO: 20 - protein, 225 aa, Homo sapiens
  2A11
AHTCPPCPAPELLGGPSVFLFPPKPKDTLMISRTPEVTYVVVDVSHEDPEVKFNWYVDGVEVHNAKTEPREEQYNSTYRVVSVFTVLHRDWLNGKEYKCKVSNKALPAPIGKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTLKSLSLSPGK

SEQ ID NO: 21 - protein, 225 aa, Homo sapiens
  2C06
AHTCPPCPAPELLGGPSVFLFPPKPKDTLMISRTPEVTCVVVDVSHEDPEVKFNWYVDGVGVHNAKTKPREEQYNSTYRVVSVLTVLHQDWLKGKEYKCKVSNKALPAPIGKTISKAKGQPQEPQVYTLPPSREEMTKNQVSPTCLVKGFYPSDIAVEWESNGQPENNYKTTPPVLDSDGSFFLYSKLTVDKSRWQQGNVFSCSVMHEALHNHYTQKSLTLSPGK

SEQ ID NO: 22 - protein, 225 aa, Homo sapiens
  3C11
AHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPSREEMTKYQVSLTCLVKGFYPSDIAVEWESNGQPENNYKTTPPVLDSDGSFFLYSKLTVDKSRWQQGNVFSCSVMHEALHNHYTQRSLSLSPGK

SEQ ID NO: 23 - protein, 225 aa, Homo sapiens
  2F01
AHTCPPCPAPELLGGPPVFLFPPKPKDTLMIPRTPEVTCVVVDVNHEDPEVKFNWYVDGVEVRNAKTKPREEQYNSTYRVVSVLTVLHQDWLSGKEYKCKVSNKALPAPIEKTISKAKGQPREPQVYTLPPSREEMTKNQVSLTCLVKGLYPSDITVEWESNGQPENDCKTTPPVLDSDGSSFLYSKLTVDKSRWQQGNVFSCSVMHKAPHNHYTQKSLSLSPGE

SEQ ID NO: 24 - protein, 225 aa, Homo sapiens
  2H06
AHTCPPCPAPELLGGPSVFLFPPKPKDTLMISRTPEVTCVVVDVSHGDPEVKFNWHVDGVEVHNAKTKPREEQYNSTYRVVSVLTVLHQDWLNGKEYKCKVSSKALPAPIEKTISKAKGQPREPQVYTLPPSREEMTKNQVSLTCLVRGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 368 bp, Homo sapiens
  1.5.3 VH domain
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcaccaccgcctacctgcagtggagcagcctgaaggcctcggacaccgccatgtattactgtgcgagacacccttcctatggttcggggagtcccaactttgactactggggccagggaaccctggtcaccgtctcctcagc

SEQ ID NO: 26 - protein, 122 aa, Homo sapiens
  1.5.3 VH domain
EVQLVQSGAEVKKPGESLKISCKGSGYSFTSYWIGWVRQMPGKGLEWMGIIYPGDSDTRYSPSFQGQVTISADKSITTAYLQWSSLKASDTAMYYCARHPSYGSGSPNFDYWGQGTLVTVSS

SEQ ID NO: 27 - DNA, 338 bp, Homo sapiens
  1.5.3 VL domain
gatattgtgatgacccagactccactctcctcacctgtcacccttggacagccggcctccatctcctgcaggtctagtcaaagcctcgtatacagtgatggaaacacctacttgagttggcttcagcagaggccaggccagcctccaagactcctaatttataagatttctaaccggttctctggggtcccagacagattcagtggcagtggggcagggacagatttcacactgaaaatcagcagggtggaagctgaggatgtcggggtttattactgcgtgcaagctacacaatttcctctcactttcggcggagggaccaaggtggagatcaaacg

SEQ ID NO: 28 - protein, 112 aa, Homo sapiens
  1.5.3 VL domain
DIVMTQTPLSSPVTLGQPASISCRSSQSLVYSDGNTYLSWLQQRPGQPPRLLIYKISNRFSGVPDRFSGSGAGTDFTLKISRVEAEDVGVYYCVQATQFPLTFGGGTKVEIK

(19) United States
(12) Patent Application Publication
(10) Pub. No.: US 2010/0184959 A1
(43) Pub. Date: Jul. 22, 2010

(54) Polypeptide Variants

(75) Inventors: Gulin Guler-Gane, Cambridge (GB); Robert George Edward Holgate, Cambridge (GB); Lutz Ulrich Jochen Wilhelm Jermutus, Cambridge (GB); Jacqueline Michaela Levens, Cambridge (GB); John Norman Lund, Cambridge (GB); Ross Anthony Stewart, Cambridge (GB); Albert George Thom, Cambridge (GB); Carl Innes Webster, Cambridge (GB)

Correspondence Address: MEDIMMUNE, LLC;Patrick Scott Alban, ONE MEDIMMUNE WAY, GAITHERSBURG, MD, 20878, US

(73) Assignee: MEDIMMUNE LIMITED, Cambridge (UK)

(21) Appl. No.: 12/532,022

(22) PCT Filed: Mar. 19, 2008
(86) PCT No.: PCT/GB08/00967

§ 371 Date: Sep. 18, 2009

Related U.S. Application Data
(60) Provisional application No. 60/895,695, filed on Mar. 19, 2007.

(51) Int. Cl. C07K 16/00; C12P 21/00; C12P 19/34

(52) U.S. Cl. 530/387.3; 435/69.6; 435/91.2

(57) ABSTRACT

The present invention relates to methods for selecting, obtaining or producing Fc variant polypeptides which show altered recognition for an Fc ligand (e.g., FcγR, CIq). Additionally, the Fc variant polypeptides may have altered antibody-dependent cell-mediated cytotoxicity (ADCC) and/or complement dependent cytotoxicity (CDC) activity. The invention further provides methods and protocols for the application of said Fc variant polypeptides particularly for therapeutic purposes.

### FIELD OF THE INVENTION

The present application relates to methods for selecting, obtaining or producing Fc variant polypeptides which show altered recognition for an Fc ligand and may also exhibit improved effector function. It further relates to manufacture and use of these variant Fc polypeptides following selection, for example in therapy.

### BACKGROUND OF THE INVENTION

An antibody molecule is made up of two identical heavy and two identical light chains held together by interchain disulfide bonds. These chains can be separated by reduction of the S—S bonds and acidification. The most abundant type of antibody in the circulation is immunoglobulin G.

Several antibody effector functions are mediated by Fc receptors (FcRs), which bind the Fc region of an antibody. FcRs are defined by their specificity for immunoglobulin isotypes and the Fc receptors for IgG antibodies are referred to as FcγR. The interaction of the Fc region with its appropriate ligand effects a variety of responses including Antibody-Dependent Cellular Cytotoxicity (ADCC) and Complement Dependent Cytotoxicity (CDC).

C1q and two serine proteases, C1r and C1s, form the complex C1, the first component of the CDC pathway. To activate the complement cascade, it is necessary for C1q to bind to at least two molecules of IgG1, IgG2 or IgG3 (IgG4 does not activate complement), but only one molecule of IgM attached to the antigenic target (Ward & Ghetie (1995) Ther. Immun. 2: 77-94).

FcγRs are part of the immunoglobulin gene superfamily. They are expressed constitutively on haemopoeitic cells such as lymphocytes, macrophages, eosinophils, neutrophils and natural killer cells and can be differentially up-regulated when the cells are exposed to activating agents such as cytokines. They have an IgG binding α-chain with an extracellular portion composed of either two (FcγRII and FcγRIII) or three (FcγRI) Ig-like domains. FcγRI and FcγRIII also have accessory proteins (γ and ζ respectively) associated with the α-chain that function in signal transduction. FcγRs have specificity for the Fc region of the gamma heavy chain of IgG (Berken & Benacerraf (1966) J. Exp. Med. 123(1): 119-44). It is thought that FcγRs evolved in parallel with IgG and thus now provide a crucial link between phagocytic effector cells and the lymphocytes that secrete IgG.

The three types of FcγR: FcγRI(CD64), II(CD32) and III(CD16) can be further classified into isoforms: FcγRIa, Ib, Ic, IIa, IIb1, IIb2, IIc, IIIa and IIIb. It is the C_{H}2 domains of the antibody, including the lower hinge region, that contain the sequences responsible for difference in binding to FcγRs. Stimulation of these receptors may result in the activation of effector functions such as ADCC, phagocytosis, superoxide burst and release of inflammatory mediators.

Yet another level of complexity is the existence of a number of FcγR polymorphisms in the human proteome. A particularly relevant polymorphism with clinical significance is V158/F158 FcγRIIIa. Human IgG1 binds with greater affinity to the V158 allotype than to the F158 allotype. Approximately 10-20% of humans are V158/V158 homozygotes.

Human FcγRIIIa is expressed on leucocytes that include Natural Killer (NK) cells, where it can mediate ADCC in response to target cells that have been sensitised with IgG antibody. Whilst the affinity of the receptor for the Fc region of monomeric IgG is only in the μM range, complexed IgG is able to bind multiple receptors with high avidity, and subsequently to trigger ADCC. The importance of the receptor for therapy with mAbs is illustrated by the treatment of follicular non-Hodgkin's lymphoma using rituximab. In this case patient survival is correlated with the allotype of FcγRIIIa, such that possession of the V158 allotype is preferable to the F158 allotype (Cartron et al., (2002) Blood 99; 754-758). Rituximab mediates ADCC more effectively via the V158 allotype than the F158 allotype (Dall'Ozzo et al (2004) Cancer Res. 64: 4664-4669). It is inferred that ADCC is a principal mechanism of action for rituximab in vivo.

Currently, many residues within the Fc sequence have been located that modulate function, including surface residues implicated by the alanine scanning mutagenesis approach (U.S. Pat. No. 6,737,056; Shields et al., (2001) J. Biol. Chem. 276: 6591-6604) and by computer modelling approaches using algorithms (US20050054832; Lazar et al., (2006) PNAS USA 103: 4005-4010). This includes the majority of the surface residues of the CH2 domain.

The alanine scanning mutagenesis approach has been employed predominantly to solvent-exposed or surface residues within the Fc region. The limitations inherent in this approach are that amino acid replacements have been made predominantly to single alanine residues only, which have a small hydrophobic and chemically inert side chain (a methyl group), and which cannot sustain electrostatic interactions or form hydrogen bonds. In addition, the contribution of numerous less exposed or buried residues has not been assessed, which residues would be anticipated to modulate function via a diffused structural change.

Computational screening methods comprising energy calculation have been used to model the interactions between the FcγRs and Fc, based on the solved crystal structures for FcγRIIIb interaction with Fcγ1 (Sondermann et al., (2000) Nature 406: 267-273; Radaev et al., (2001) J. Biol. Chem. 276: 16469-16477). Of particular help in this in silico approach is that there is a conserved docking interaction between Trp-90 and Trp-113 on the FcγR and Pro-329 on the CH2 domain of IgG-Fc. The algorithm has been applied primarily to variants within the CH2 domain in the case of the FcγR, presumably due to the difficulties inherent in modeling diffused structural changes arising from variants within the CH3 domains and having replacements distant from the binding site for FcγR within the CH2 domain. The approach is restricted to the homologous family of FcγR, and does not extend to the C1q-Fcγ1 interaction, which currently lacks a high-resolution complex structure.

An approach to express IgG in a CHO cell line that lacks the glycosylation enzyme α1,6-fucosyltransferase, leads to IgG with complex oligosaccharide chains without fucose residues (i.e. afucosylated). Afucosylated IgG demonstrates enhanced ADCC with human peripheral blood mononuclear cells (Niwa et al (2004) Cancer Res. 64: 2127-2133). The use of afucosylated IgG is likely to be applicable only to those Fcγ receptors having a glycosylation site at the FcγR-Fcγ1 interface at Asn-162 (FcγRIIIa and FcγRIIIb) (Ferrara et al., (2006) J. Biol. Chem. 281: 5032-5036), and is thus not likely to be applicable to the generation of variant IgG antibodies having enhanced recognition for FcγRI, FcγRIIa, FcγRIIb or other effector ligands not homologous with the FcγR such as FcRn or C1q.

In addition to the above techniques, a yeast surface display library has been employed in order to generate glycosylated IgG1 variants having enhanced binding for FcγRIIIa (US20050064514). A disadvantage of this approach is the upper limit, at around 10 million, to the number of independent Fcγ1 variants that can be explored by the approach, limiting the segment of sequence space that can be searched.

An embodiment of the present invention employs the use of ribosome display technology incorporating in vitro translation and covalent or non-covalent linkage between genotype, such as RNA, and the encoded phenotype, such as a variant polypeptide of interest, to select for variant polypeptides that have altered recognition of an Fc ligand compared with a parent Fc polypeptide.

Ribosome or polysome display and selection involves construction of nucleic acid libraries, screening for binding, and identification of binding entities of interest. The library is made by synthesising a DNA pool of diverse sequences that are then transcribed to produce a pool of mRNAs. In vitro translation is used to generate the encoded polypeptides or proteins displayed, and desirable binding interactions are selected using immobilised target antigen. mRNA encoding the binding entities can be used to make cDNA, which can then be amplified and the process may be repeated to enrich the population for genes encoding binders. The selected proteins may later be identified by cloning individual coding sequences and DNA sequencing.

The technology has been reviewed extensively (Hanes et al., (2000) Meth. Enzymol. 328: 403-430; Plückthun et al., (2000) Adv. Prot. Chem. 55: 367-403; Lipovsek and Plückthun (2004) J. Immunological Methods 290: 51-67). The technology has been used for the display of antibody fragments, peptides and various proteins, including periplasmic and cytoplasmic proteins such as β-lactamase and ankyrin-repeat proteins.

Recovery of mRNA from polysome complexes was first reported in 1973 in a paper describing a protocol to capture mRNA coding for a mouse immunoglobulin L-chain using antibodies and immobilised oligothymidine (Schechter (1973) PNAS USA 70: 2256-2260). Improvements to the polysome immunoprecipitation protocols were made by Payvar and Schimke (1979, Eur. J. Biochem. 101: 271-282) and cDNA clones for the heavy chain of HLA-DR antigens were obtained after immunoprecipitation of polysomes using a monoclonal antibody (Korman et al., (1982) PNAS USA 79: 1844-1848). Production of libraries of antibodies by ribosome display was proposed and patented by Kawasaki (U.S. Pat. No. 5,643,768, U.S. Pat. No. 5,658,754 and EP0494955B).

There have been various examples of the use of ribosome display using either eukaryotic or prokaryotic translation systems. The first demonstration of selection of peptide ligands using an E. coli extract was by Mattheakis et al., (1994, PNAS USA 91: 9022-9026; 1996, Methods Enzymol 267: 195-207). This group demonstrated selection of peptide ligands that are similar to known peptide epitopes of a given antibody, using the antibody as a selection substrate. High-affinity peptide ligands which bind prostate-specific antigen have been identified using polysome selection from peptide libraries using a wheat germ extract translation system (Gersuk et al., (1997) Biotech and Biophys. Res. Com. 232: 578-582). The selection of functional antibody fragments was reported using an E. coli translation system designed for increased yield of ternary complexes and allowing disulphide bond formation (Hanes and Pluckthun, (1997) PNAS USA 94: 4937-4942). This experimental set up has subsequently been used to select antibodies from a murine library, and it was shown that affinity maturation occurs during the selection due to the combined effect of PCR errors and selection. A scFv fragment with a dissociation constant of about 10⁻¹¹M was obtained (Hanes et al., (1998) PNAS USA 95: 14130-50). Enrichment for specific binding fragments of antibodies from mixed populations using rabbit reticulyocyte lysate extracts has also been demonstrated (He and Taussig (1997) Nucleic Acids Res. 25: 5132-5234).

mRNA display, like ribosome display, uses a complex between mRNA and the encoded polypeptide as the basic selection unit. What distinguishes mRNA display from ribosome display is the covalent nature of the linkage between the mRNA and the protein. The linkage is achieved through a small adaptor molecule, typically puromycin (Nemoto et al., (1997) FEBS Lett 414: 405; Roberts and Szostak, (1997) PNAS USA 94: 12297; Takahashi et al., (2003) Trends Biochem. Sci. 28: 159). mRNA display is not limited to 4° C., which is the usual temperature at which ribosome display is carried out. Typically the temperature at which selections are performed is limited by the stability of the protein target. mRNA display has been used for affinity selections of peptides and antibodies (Reviewed in Lipovsek and Plückthun, (20.04) J. Immunol. Methods 290: 51-97).

In an embodiment of the present invention, ribosome display is applied to select for Fc polypeptide variants with altered recognition of an Fc ligand, for example FcγRIIIa and C1q, compared with a parent Fc polypeptide. With regard to the Fc receptor, one of the challenges of this undertaking is the very low affinity of the receptor for the monomeric IgG ligand. For FcγRIIIa, the affinity is between 0.5 to 3 μM (K_{D}) (Okazaki, (2004) J. Mol. Biol. 336; 1239-1249). In particular, when using ribosome display for this purpose, Fcγ1 (IgG1Fc) is expressed in vitro in an E. coli bacterial extract and consequently is aglycosylated. This is in contrast to previous methods, as discussed above, for the generation of Fc variants where Fcγ1 is expressed in a glycosylated form. Using aglycosylated Fcγ1 has the effect of lowering the anticipated affinity by approximately 100 fold relative to glycosylated IgG, giving a K_{D} in the range 0.1 mM. In addition, within the context of ribosome display there is no direct evidence for dimer formation between heavy chains within the Fc region. A lack of dimer formation would be expected to result in an additional reduction in affinity by multiple orders of magnitude, since the co-crystal structure indicates that both heavy chains within the Fc homo-dimer interact with FcγRIII (Sondermann, (2000) Nature 406: 267-273). In summary, given the very high K_{D} anticipated for ribosome display between Fcγ1 and FcγRIIIa, it is surprising that the selections to identify suitable Fc variant polypeptides with this technique, can proceed successfully.

Ribosome display can also be used to generate Fc variants that bind to other Fc receptors. Alternatively, Fc variants that recognise FcγRIIIa, generated in the present application, can be tested for recognition of additional Fc receptors such as FcγRIIb, since these receptors share high sequence homology with each other.

A similar argument, for an expected lack of success when using ribosome display for generating Fc variants that bind to Fc ligands, can be applied for C1q. The affinity of monomeric IgG for binding to C1q as determined by ultracentrifuge studies is in the region of mM (K_{D})(Schumaker V. N. et al., (1976) Biochemistry 15: 5175-5181). Therefore, given the very high K_{D} anticipated for ribosome display between Fcγ1 and C1q, it is again surprising that selections can proceed successfully.

### DESCRIPTION OF THE FIGURES

FIGS. 1(a), (b), (c) and (d) show data for a selection of Fc polypeptide variants (as Fcγ1-FLAG variants) in an AlphaScreen FcγRIIIa inhibition assay. FIGS. 1(a) to (d) show data for the V158 allotype of FcγRIIIa. These assays were performed upon protein generated in E. coli i.e. the Fc-FLAG preparations are bacterial and are therefore aglycosylated.

FIG. 2 shows the binding of a selection of Fc polypeptide variants in IgG1 format to FcγRIIIa F158 allotype, by ELISA.

FIG. 3 shows ADCC from a selection of Fc polypeptide variants directed against Daudi B Cells, normalised to that with commercial rituximab (V/F allotype NK effector cells at 25:1 E:T).

FIG. 4 shows the binding of a selection of Fc polypeptide variants in IgG1 format to C1q, by ELISA.

FIG. 5 shows the binding of a selection of Fc polypeptide variants in IgG1 format to FcγRIIb, by ELISA.

FIGS. 6 (a), (b), (c), (d) and (e) show the amino acid sequence alignment of the Wild Type Fc region and Fc polypeptide variants disclosed herein. The numbering shown in the figures is that of the EU index defined by Kabat et al., the amino acid substitutions are boxed, and the number preceding the clone ID is the SEQ ID NO. corresponding to that sequence.

FIGS. 7 (a), (b), (c), (d), (e), (f), (g), (h), (i), and (j) represent the oligosaccharide structures found on antibodies expressed in mammalian cells. The abbreviations used for each structure are indicated to the right.

FIG. 8 shows ADCC response to Fc polypeptide variants in IgG1 format. The variants comprise a point mutation at residue 243 compared to wild-type anti-CD20 antibody. The effector to target ratio was 25:1, with a 158FcγRIIIa F/F allotype Donor. The target cells were Daudi cells.

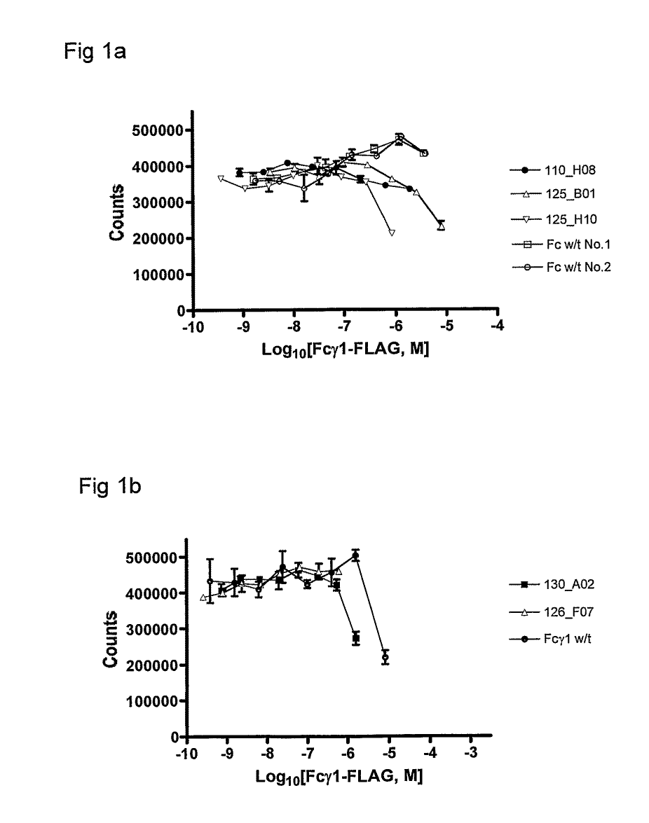

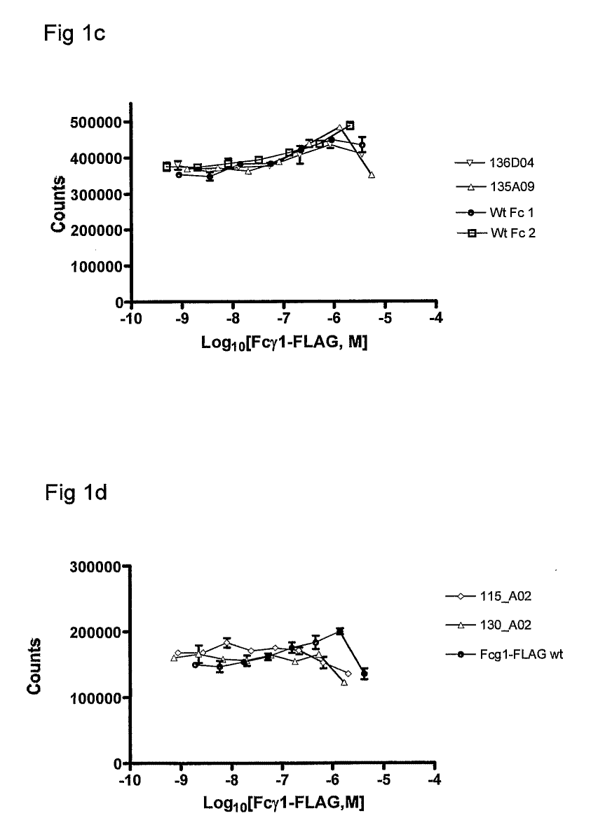

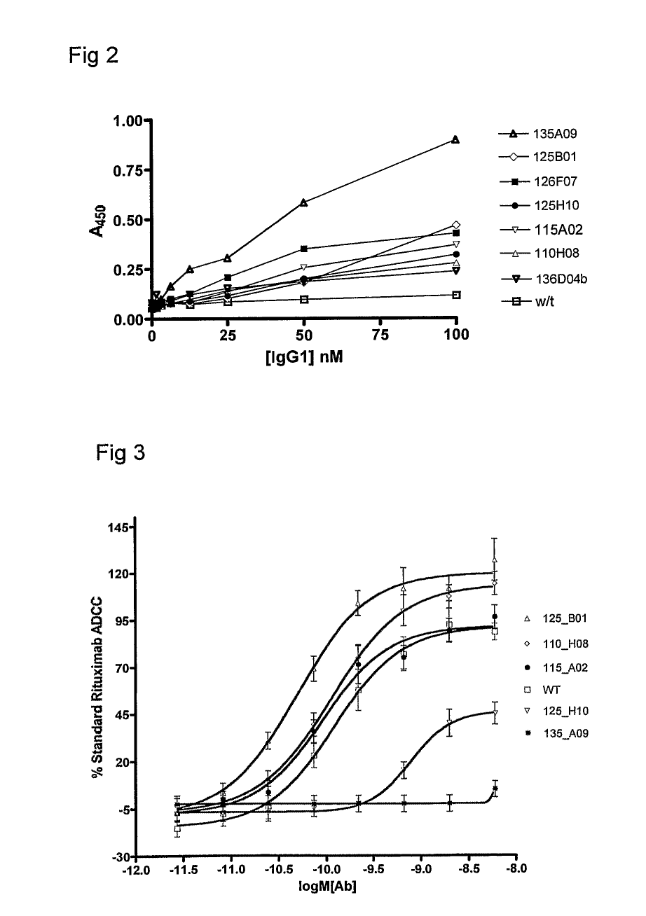

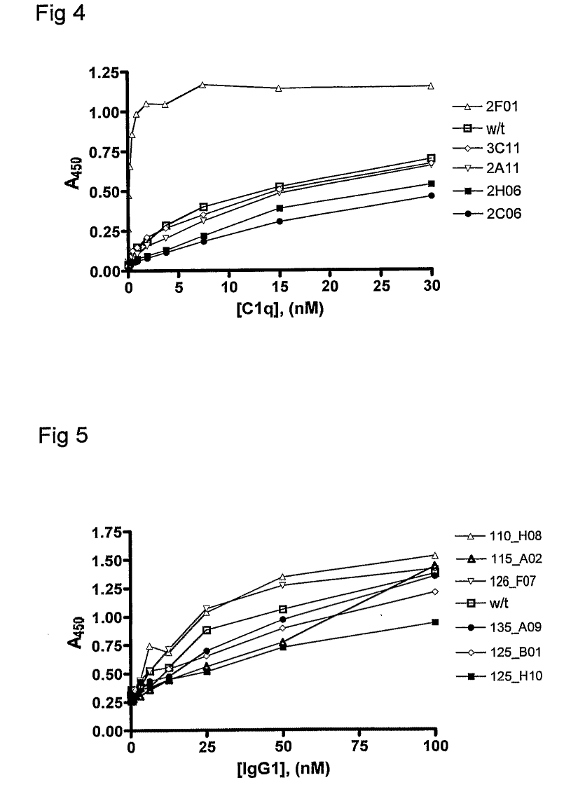

### SUMMARY

According to one embodiment of the present invention there is provided a method of providing an Fc polypeptide variant with altered recognition of an Fc ligand and/or improved effector function compared with a parent Fc polypeptide, the method comprising:
(a) providing mRNA molecules, each mRNA molecule comprising a nucleotide sequence encoding an Fc polypeptide variant and lacking an in-frame stop codon;
(b) incubating the mRNA molecules under conditions for ribosome translation of the mRNA molecules to produce encoded Fc polypeptide variants, whereby complexes each comprising at least mRNA and encoded Fc polypeptide variant are formed;
(c) bringing the complexes into contact with an Fc ligand that binds the parent Fc polypeptide, and selecting one or more complexes each displaying an Fc polypeptide variant able to bind the Fc ligand under the conditions of the selection; and
(d) determining recognition or effector function of selected Fc polypeptide variant or variants, whereby one or more Fc polypeptide variants with altered Fc ligand recognition and/or improved effector function compared with the parent Fc polypeptide are obtained.

Preferably the complexes formed in step (c) above comprise mRNA, the encoded Fc polypeptide variant and ribosome.

Fc ligand recognition may be determined by comparing the ability of selected Fc polypeptide variant or variants and parent Fc polypeptide to bind Fc ligand using one or more assays known in the art, including but not limited to, radio immunoassay (RIA) and/or ELISA.

In certain embodiments of the invention, the Fc ligand is an Fc receptor. The Fc receptor may be an Fcγ receptor, such as a receptor selected from FcγRI, FcγRII or FcγRIII families. In one embodiment, the receptor is FcγRIIb and the Fc polypeptide variant of the invention shows reduced binding to FcγRIIb. In an alternative embodiment, the receptor is FcγRIIIa, which may be selected from the group consisting of V158 or F158 allotype of FcγRIIIa. In a specific embodiment, the Fc polypeptide variant of embodiments of the invention shows improved binding to FcγRIIIa. In another specific embodiment, the Fc polypeptide variant retains equal binding to either V158 or F158 allotype of FcγRIIIa.

In another embodiment, the Fc ligand is C1q and the Fc polypeptide variant of embodiments of the invention shows improved binding to C1q. In still another embodiment, the Fc ligand is C1q and the Fc polypeptide variant of embodiments of the invention shows reduced binding to C1q.

In a method according to embodiments of the invention, the Fc polypeptide variant may comprise a variant human IgG Fc region. This may be selected from IgG1, IgG2, IgG3 or IgG4. In a specific embodiment, the IgG region is IgG1.

Effector function of the Fc polypeptide variants of embodiments of the invention may be determined by comparing the ability of selected variants in IgG format with that of the parent Fc polypeptide, also in IgG format, in an ADCC or CDC assay. In certain embodiments, the Fc polypeptide variants of the present embodiments show improved effector function in said assays when compared with a parent Fc polypeptide. In other embodiments the Fc polypeptide variants of the present embodiments show reduced effector function in said assays when compared with a parent Fc polypeptide.

In an embodiment of the present invention, the Fc polypeptide variant may be prepared in IgG format with the variable regions of an anti-CD20 antibody. These heavy and light chain variable regions may be selected from a panel of anti-CD20 antibodies as given in Table 1 in International Patent Application WO 06/130458, herein incorporated by reference. In a specific embodiment of the present invention, the Fc polypeptide variant may be prepared in IgG format with the variable regions of the anti-CD20 antibody 1.5.3 as disclosed in Table 1 of International Patent Application WO 06/130458. The nucleic acid and amino acid sequences of the heavy and light chain variable regions respectively have SEQ ID NOS: 25 to 28.

In a method according to embodiments of the invention mRNA retrieved from a selected complex displaying a selected Fc polypeptide variant may be amplified and copied into DNA or cDNA encoding the selected Fc polypeptide variant. DNA may be provided in an expression system for production of a product, which product is or comprises the selected Fc polypeptide variant or a polypeptide chain of the selected Fc polypeptide variant. Embodiments of the invention may further comprise isolating or purifying the product, which may be formulated into a composition comprising at least one additional component.

DNA encoding the selected Fc polypeptide variant or a polypeptide chain of the selected Fc polypeptide variant may be provided within a nucleotide sequence to provide a DNA sequence encoding a fusion protein comprising the selected Fc polypeptide variant, or a polypeptide chain of the selected Fc polypeptide variant, fused to additional amino acids. DNA comprising said nucleotide sequence encoding said fusion protein may be provided in an expression system for production of a product, which product is the fusion protein. Embodiments of the invention may further comprise isolating or purifying such product, which may be formulated into a composition comprising at least one additional component.

In some embodiments, the parent Fc polypeptide is an antibody molecule, or immunoadhesion. In certain embodiments, a parent Fc polypeptide comprises an antibody binding domain (e.g., an scFv, VH, Fd (consisting of the VH and CH1 domains), dAb molecule) fused to the Fc domain of an antibody molecule (consisting of CH2 and CH3 domains). This polypeptide can incorporate a single Fc chain (CH2 & CH3) or a dimeric Fc chain (CH2-CH3-linker-CH2-CH3 [scFc]), where the linker will allow correct folding of the two Fc domains.

In other embodiments, non-antibody polypeptides are employed, and these may include receptors, enzymes, peptides and protein ligands.

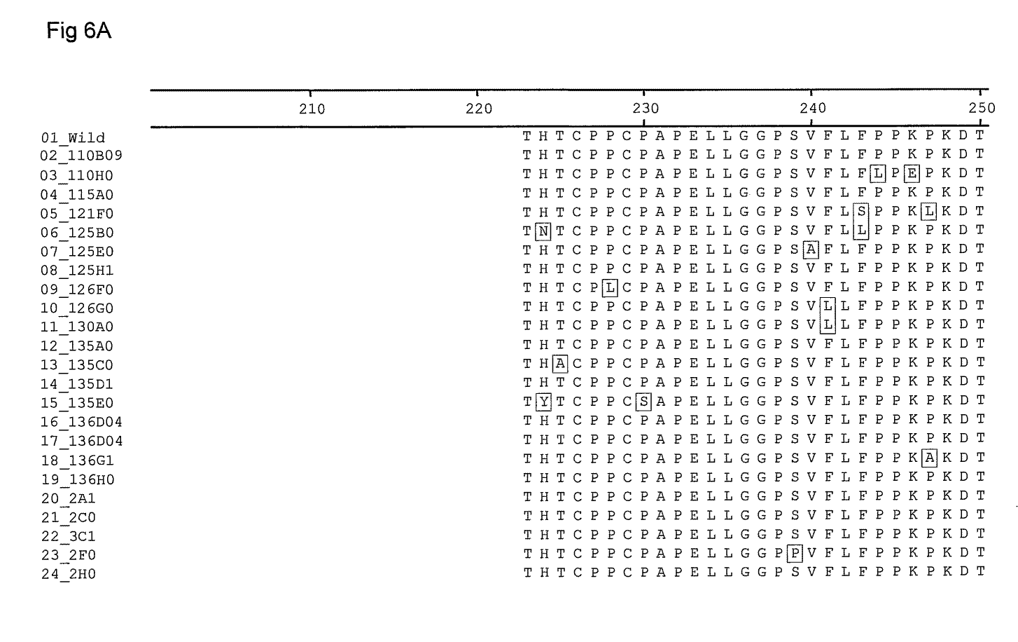

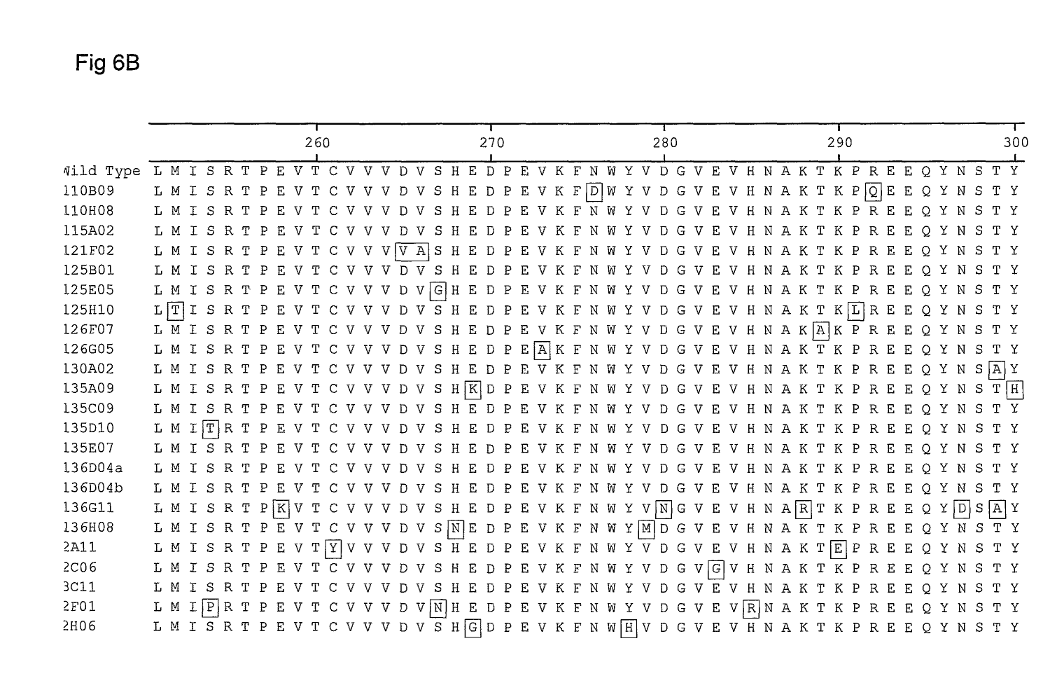

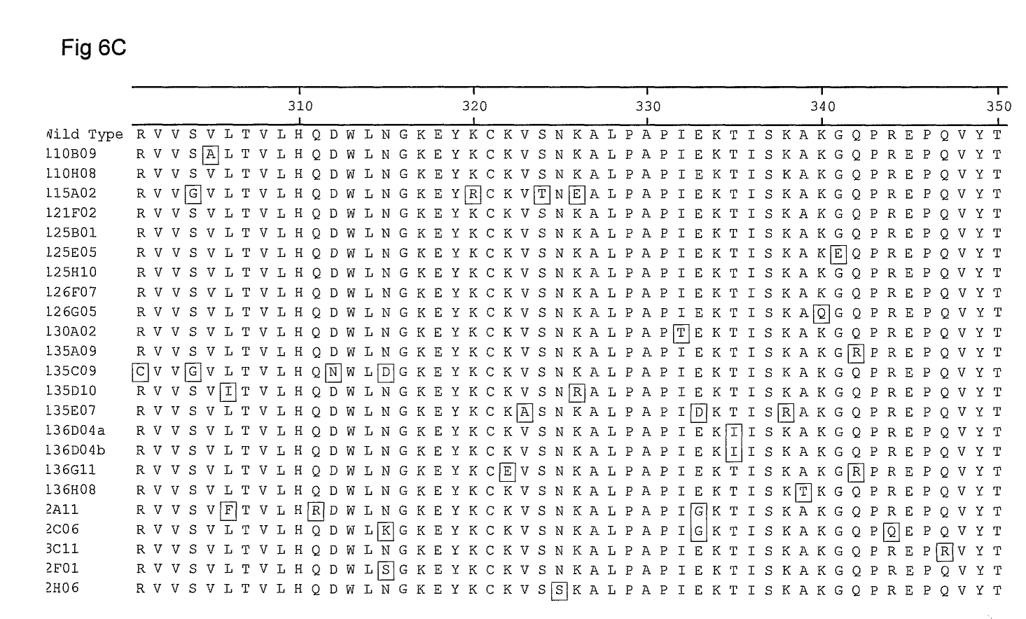

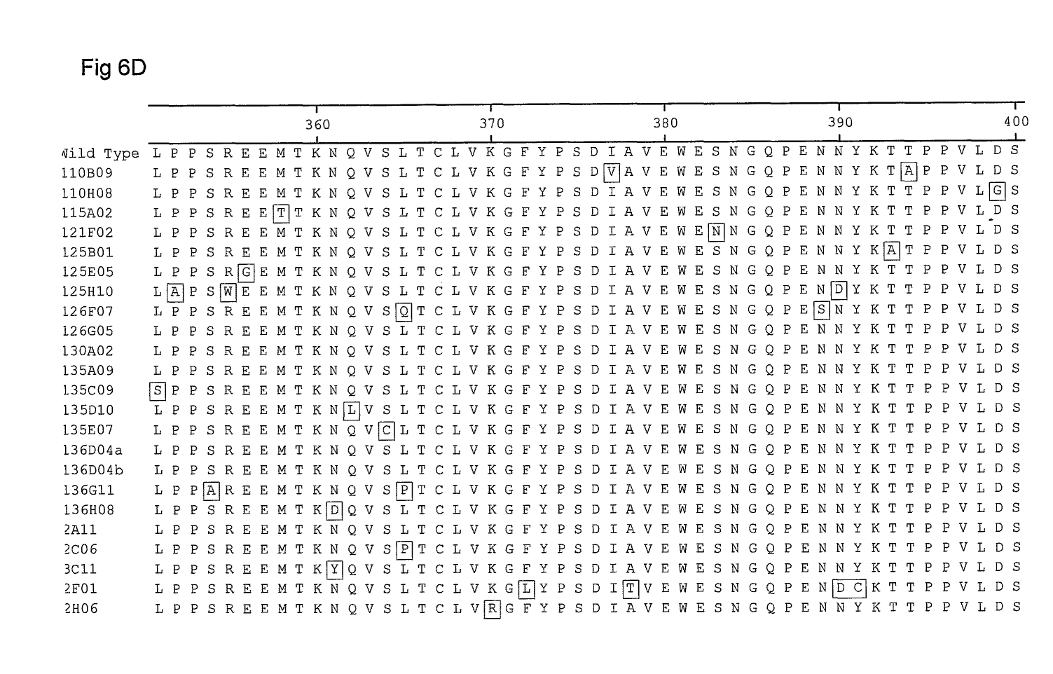

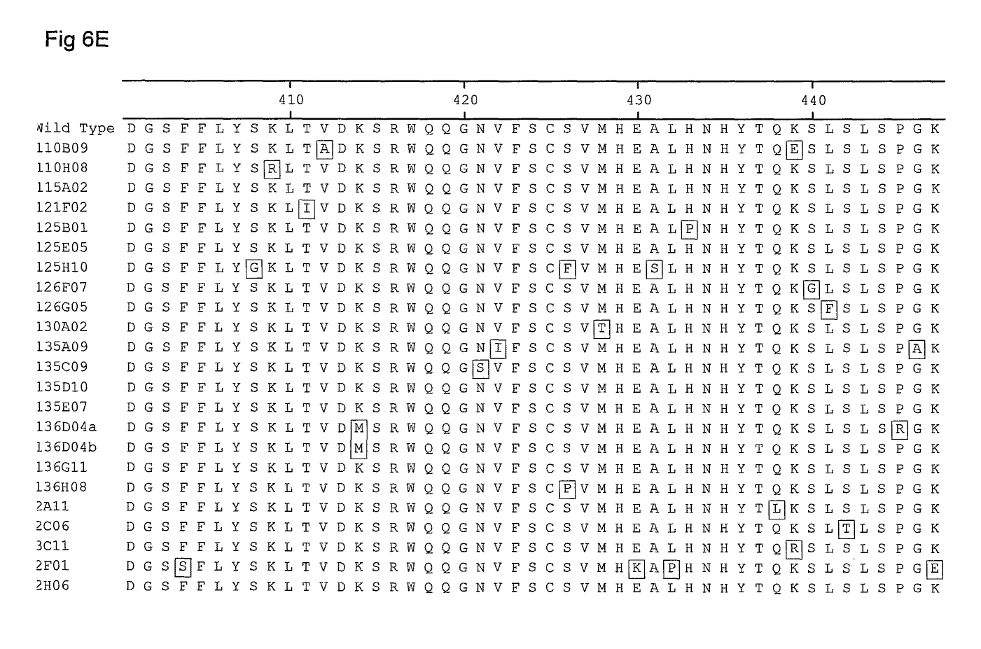

Embodiments of the present invention are illustrated by methods wherein the parent Fc polypeptide is a wild-type Fc polypeptide. In particular, the parent Fc polypeptide is human wild-type Fc polypeptide of the F allotype, having the amino acid sequence shown in SEQ ID NO: 1. Embodiments of the invention may provide an Fc polypeptide variant having a mutation at one or more of the following positions in the amino acid sequence of SEQ ID NO: 1, as represented in FIG. 6, to the residue as indicated below: 224N/Y, 225A, 228L, 230S, 239P, 240A, 241L, 243S/L/G/H/I, 244L, 246E, 247L/A, 252T, 254T/P, 258K, 261Y, 265V, 266A, 267G/N, 268N, 269K/G, 273A, 276D, 278H, 279M, 280N, 283G, 285R, 288R, 289A, 290E, 291L, 292Q, 297D, 299A, 300H, 301C, 304G, 305A, 306I/F, 311R, 312N, 315D/K/S, 320R, 322E, 323A, 324T, 325S, 326E/R, 332T, 333D/G, 335I, 338R, 339T, 340Q, 341E, 342R, 344Q, 347R, 351S, 352A, 354A, 355W, 356G, 358T, 361D/Y, 362L, 364C, 365Q/P, 370R, 372L, 377V, 378T, 383N, 389S, 390D, 391C, 393A, 394A, 399G, 404S, 408G, 409R, 411I, 412A, 414M, 421S, 422I, 426F/P, 428T, 430K, 431S, 432P, 433P, 438L, 439E/R, 440G, 441F, 442T, 445R, 446A, 447E, wherein the numbering of the residues is that of the EU index as in Kabat.

Where a mutation to more than one amino acid at a specific position is possible, this is indicated by the nomenclature used above, for example 247L/A, where the mutation at position 247 can be to either L (leucine) or A (alanine).

Methods described herein provide a Fc polypeptide variant capable of binding to an FcγRIIb or FcγRIIIa comprising a set of mutations in the human wild-type sequence of SEQ ID NO: 1, as represented in FIG. 6, selected from the group consisting of the following sets of mutations:

| SEQ ID No | Clone ID | Substitution^{a} |
|---|---|---|
| 2 | 110B09 | N276D, R292Q, V305A, I377V, |
| | | T394A, V412A, K439E |
| 3 | 110H08 | P244L, K246E, D399G, K409R |
| 4 | 115A02 | S304G, K320R, S324T, K326E, M358T |
| 5 | 121F02 | F243S, P247L, D265V, V266A, |
| | | S383N, T411I |
| 6 | 125B01 | H224N, F243L, T393A, H433P |
| 7 | 125E05 | V240A, S267G, G341E, E356G |
| 8 | 125H10 | M252T, P291L, P352A, R355W, |
| | | N390D, S408G, S426F, A431S |
| 9 | 126F07 | P228L, T289A, L365Q, N389S, S440G |
| 10 | 126G05 | F241L, V273A, K340Q, L441F |
| 11 | 130A02 | F241L, T299A, I332T, M428T |
| 12 | 135A09 | E269K, Y300H, Q342R, V422I, G446A |
| 13 | 135C09 | T225A, R301C, S304G, D312N, |
| | | N315D, L351S, N421S |
| 14 | 135D10 | S254T, L306I, K326R, Q362L |
| 15 | 135E07 | H224Y, P230S, V323A, E333D, |
| | | K338R, S364C |
| 16 | 136D04a | T335I, K414M, P445R |
| 17 | 136D04b | T335I, K414M, |
| 18 | 136G11 | P247A, E258K, D280N, K288R, |
| | | N297D, T299A, K322E, Q342R, |
| | | S354A, L365P |
| 19 | 136H08 | H268N, V279M, A339T, N361D, S426P |

^{a}Wherein the numbering of the residues is that of the EU index as in Kabat.

Methods described herein also provide a Fc polypeptide variant capable of binding to C1q comprising a set of mutations in the human wild-type sequence of SEQ ID NO: 1, as represented in FIG. 6, selected from the group consisting of the following sets of mutations:

| SEQ ID No | Clone ID | Substitution^{a} |
|---|---|---|
| 20 | 2A11 | C261Y, K290E, L306F, Q311R, |
| | | E333G, Q438L |
| 21 | 2C06 | E283G, N315K, E333G, R344Q, |
| | | L365P, S442T |
| 22 | 3C11 | Q347R, N361Y, K439R |
| 23 | 2F01 | S239P, S254P, S267N, H285R, |
| | | N315S, F372L, A378T, N390D, |
| | | Y391C, F404S, E430K, L432P, K447E |
| 24 | 2H06 | E269G, Y278H, N325S, K370R |

^{a}Wherein the numbering of the residues is that of the EU index as in Kabat.

Throughout this specification and claims, the numbering of the amino acid residues in the heavy chain of the immunoglobulin is that of the EU index as defined by Kabat et al (Sequences of proteins of immunological interest, 5^{th} Ed., U.S. Dept. of Health and Human Services, NIH, 1991 and later editions).

A Fc polypeptide variant according to embodiments of the invention may be provided to contain one or more additional changes compared with a starting or parent Fc polypeptide, which may be a wild-type or native protein or a previously obtained polypeptide variant. A number of different modifications to Fc polypeptides are known (both naturally occurring mutants and artificially created variants) with modified properties compared with wild-type. One or more of these properties may be retained or provided in a Fc polypeptide variant according to present embodiments.

A further embodiment of the present invention, provides an Fc polypeptide variant having a mutation at two or more positions in the amino acid sequence of human wild-type sequence of SEQ ID NO: 1, wherein the residue provided at any one of said positions is selected from the following: 224N/Y, 225A, 228L, 230S, 239P, 240A, 241L, 243S/L/G/H/I, 244L, 246E, 247L/A, 252T, 254T/P, 258K, 261Y, 265V, 266A, 267G/N, 268N, 269K/G, 273A, 276D, 278H, 279M, 280N, 283G, 285R, 288R, 289A, 290E, 291L, 292Q, 297D, 299A, 300H, 301C, 304G, 305A, 306I/F, 311R, 312N, 315D/K/S, 320R, 322E, 323A, 324T, 325S, 326E/R, 332T, 333D/G, 335I, 338R, 339T, 340Q, 341E, 342R, 344Q, 347R, 351S, 352A, 354A, 355W, 356G, 358T, 361D/Y, 362L, 364C, 365Q/P, 370R, 372L, 377V, 378T, 383N, 389S, 390D, 391C, 393A, 394A, 399G, 404S, 408G, 409R, 411I, 412A, 414M, 421S, 422I, 426F/P, 428T, 430K, 431S, 432P, 433P, 438L, 439E/R, 440G, 441F, 442T, 445R, 446A, 447E, wherein the variant has altered recognition of an Fc ligand and/or improved effector function compared with a parent Fc polypeptide, and wherein the numbering of the residues is that of the EU index as in Kabat.

In an embodiment of the present invention, at least one of the mutations may be located in a CH3 domain of the human wild-type sequence of SEQ ID NO: 1.

In a preferred embodiment of the present invention, an Fc polypeptide variant is provided which comprises a set of mutations in the human wild-type sequence of SEQ ID NO: 1 selected from the group consisting of the following sets of mutations:

### (2) N276D, R292Q, V305A, I377V, T394A, V412A and K439E;

### (3) P244L, K246E, D399G and K409R;

### (4) S304G, K320R, S324T, K326E and M358T;

### (5) F243S, P247L, D265V, V266A, S383N and T411I;

### (6) H224N, F243L, T393A and H433P;

### (7) V240A, S267G, G341E and E356G;

### (8) M252T, P291L, P352A, R355W, N390D, S408G, S426F and A431S;

### (9) P228L, T289A, L365Q, N389S and S440G;

### (10) F241L, V273A, K340Q and L441F;

### (11) F241L, T299A, I332T and M428T;

### (12) E269K, Y300H, Q342R, V422I and G446A;

### (13) T225A, R301C, S304G, D312N, N315D, L351S and N421S;

### (14) S254T, L306I, K326R and Q362L;

### (15) H224Y, P230S, V323A, E333D, K338R and S364C;

### (16) T335I, K414M and P445R;

### (17) T335I and K414M;

### (18) P247A, E258K, D280N, K288R, N297D, T299A, K322E, Q342R, S354A and L365P;

### (19) H268N, V279M, A339T, N361D and S426P;

### (20) C261Y, K290E, L306F, Q311R, E333G and Q438L;

### (21) E283G, N315K, E333G, R344Q, L365P and S442T;

### (22) Q347R, N361Y and K439R;

### (23) S239P, S254P, S267N, H285R, N315S, F372L, A378T, N390D, Y391C, F404S, E430K, L432P and K447E; and

### (24) E269G, Y278H, N325S and K370R;

wherein the numbering of the residues is that of the EU index as in Kabat.

Each number in parentheses preceding the set of mutations indicates the SEQ ID NO attributed to the Fc polypeptide variant sequence having that set of mutations. As such, an embodiment of the present invention provides an Fc polypeptide variant having an amino acid sequence selected from SEQ ID NOs: 2 to 24 and nucleic acid sequences encoding these.

An Fc polypeptide variant of an embodiment of the invention may comprise a sequence with ten or fewer, preferably four, five, six, seven or eight substitutions relative to a parent Fc polypeptide.

Further aspects and embodiments of the present invention are disclosed herein in and preferred aspects and embodiments are subject to the claims included below.

### DEFINITIONS

A ‘parent Fc polypeptide’ is an Fc polypeptide comprising an amino acid sequence which lacks one or more of the Fc region alterations disclosed herein and which differs in recognition or effector function compared to a polypeptide variant as herein disclosed. The parent Fc polypeptide may comprise a native sequence Fc region or an Fc region with pre-existing amino acid sequence modifications (such as additions, deletions and/or substitutions).

A ‘wild-type Fc polypeptide’ comprises an amino add sequence identical to the amino acid sequence of an Fc region found in nature. Native human Fc polypeptides include a native sequence human IgG1 Fc region (non-A and A allotypes) and a native sequence human IgG2, IgG3 or IgG4 Fc region, as well as naturally occurring variants thereof. The human wild-type Fc polypeptide sequence of SEQ ID NO: 1 is of the F allotype.

The term ‘Fc polypeptide’ refers to any polypeptide including, but not limited to, an antibody or immunoadhesion, which comprises or consists essentially of an Fc region.

An ‘Fc polypeptide variant’ comprises an amino acid sequence which differs from that of a native sequence Fc region by virtue of more than one amino acid alterations. In certain embodiments, the Fc polypeptide variant has more than one amino acid substitution compared to a native sequence Fc region or to the Fc region of a parent Fc polypeptide, e.g. from about two to about ten amino acid substitutions, and preferably from about two, three or four to about five amino acid substitutions, in a native sequence Fc region or in the Fc region of the parent Fc polypeptide. The Fc polypeptide variant herein will, in some embodiments, possess at least about 80% homology with a native sequence Fc region and/or with an Fc region of a parent Fc polypeptide, or at least about 90% homology therewith, or at least about 95% homology therewith. ‘Homology’ can be defined as the percentage of residues in the polypeptide variant that are identical after aligning the sequences and introducing gaps, if necessary, to achieve the maximum percent homology. Methods and computer programs for the alignment are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet at al., Nuc. Acids Res. 16:10881-90, 1988; Huang et al. Computer Appls. Biosci. 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biotechnology Information (NCBI, Bethesda, Md.) and on the Internet, for use in connection with the sequence analysis programs blastp, blastn, blastx, tblastn and tblastx. Each of these sources also provides a description of how to determine sequence identity using this program. For comparisons of amino acid sequences of greater than about 30 amino acids, the Blast 2 sequences function can be employed using the default BLOSUM62 matrix set to default parameters, (gap existence cost of 11, and a per residue gap cost of 1).

Homologous sequences are typically characterized by possession of at least 60%, 70%, 75%, 80%, 90%, 95% or at least 98% sequence identity counted over the full length alignment with a sequence using the NCBI Blast 2.0, gapped blastp set to default parameters. Queries searched with the blastn program are filtered with DUST (Hancock and Armstrong, Comput. Appl. Biosci. 10:67-70, 1994). It will be appreciated that these sequence identity ranges are provided for guidance only; it is entirely possible that strongly significant homologs could be obtained that fall outside of the ranges provided.

A Fc polypeptide variant with ‘altered recognition’ of an Fc ligand may have improved or reduced FcR binding compared to a parent Fc polypeptide or to a polypeptide comprising a wild-type Fc polypeptide. The Fc polypeptide variant which displays improved binding to an FcR binds at least one FcR with better affinity than the parent Fc polypeptide. The Fc polypeptide variant which displays reduced binding to an FcR, binds at least one FcR with worse affinity than a parent Fc polypeptide. Such Fc polypeptide variants which display reduced binding to an FcR may possess little or no appreciable binding to an FcR, e.g., 0-20% binding to the FcR compared to a native sequence IgG Fc region, e.g. as determined in the Examples a polypeptide comprising wild-type Fc polypeptide. herein. The Fc polypeptide variant which displays improved binding to an FcR, is one which binds any one or more of the above identified FcRs with substantially better binding affinity than the parent Fc polypeptide, when the amounts of Fc polypeptide variant and parent Fc polypeptide in the binding assay are essentially the same. In an alternative embodiment, an Fc polypeptide variant with altered recognition of an Fc ligand may have improved or reduced binding to C1q compared to a parent Fc polypeptide or to a polypeptide comprising wild-type Fc polypeptide. In certain embodiments, an Fc polypeptide variant with altered recognition of an Fc ligand may have altered binding to one or more FcRs and altered binding to C1q compared to a parent Fc polypeptide or to In other embodiments, an Fc polypeptide variant with altered recognition of an Fc ligand may have may have improved binding to some Fc ligands and reduced binding to other Fc ligands. Fc ligand recognition may be determined, for example, by comparing the ability of selected Fc polypeptide variants of embodiments of the invention and parent Fc polypeptide to bind Fc ligand using radio immunoassay (RIA) and/or ELISA. For example, an Fc polypeptide variant with improved FcR binding may display from about 1.10 fold to about 100 fold, e.g. from about 1.15 fold to about 50 fold improvement in FcR binding affinity compared to the parent Fc polypeptide, where FcR binding affinity is determined, e.g. as disclosed in the Examples herein.

The term ‘Fc ligand’ refers to a protein capable of binding an Fc polypeptide. In certain embodiments, the Fc polypeptide is an Fc polypeptide variant of present invention. The Fc ligand may be an Fc receptor (FcR) or the protein C1q. It is specifically contemplated that an FcR is a native sequence human FcR. In particular, an FcR is one which binds an IgG antibody (a gamma receptor; FcγR) and includes receptors of the FcγRI, FcγRII, and FcγRIII subclasses, including allelic variants and alternatively spliced forms of these receptors. FcγRII receptors include FcγRIIa (an activating receptor) and FcγRIIb (an inhibiting receptor), which have similar amino add sequences that differ primarily in the cytoplasmic domains thereof. Activating receptor FcγRIIa contains an immunoreceptor tyrosine-based activation motif (ITAM) in its cytoplasmic domain. Inhibiting receptor FcγRIIb contains an immunoreceptor tyrosine-based inhibition motif (ITIM) in its cytoplasmic domain (see review in Daeron, (1997) Annu. Rev. Immunol. 15: 203-234). FcRs are reviewed in Ravetch and Kinet (1991, Annu. Rev. Immunol 9: 457-92); Capel et al., (1994, Immunomethods 4: 25-34); and de Haas et al., (1995, J. Lab. Clin. Med. 126: 330-41). Other FcRs, including those to be identified in the future are encompassed by the term ‘FcR’ herein. The term also includes the neonatal receptor, FcRn, which is responsible for the transfer of maternal IgGs to the fetus (Guyer et al., (1976) J. Immunol. 117: 587 and Kim et al., (1994) J. Immunol. 24: 249). As discussed above, C1q and two serine proteases, C1r and C1s, form the complex C1, the first component of the CDC pathway. To activate the complement cascade, it is necessary for C1q to bind to at least two molecules of IgG1, IgG2 or IgG3 but only one molecule of IgM attached to the antigenic target.

The term ‘Fc region’ is used to define a C-terminal region of an immunoglobulin heavy chain and may be a native sequence Fc region or a variant Fc region. Although the boundaries of the Fc region of an immunoglobulin heavy chain might vary, the human IgG heavy chain Fc region is usually defined to stretch from an amino acid residue at position Cys226 to the carboxyl-terminus thereof. The Fc region of an immunoglobulin generally comprises two constant domains, CH2 and CH3. The ‘CH2 domain’ of a human IgG Fc region usually extends from about amino acid 231 to about amino acid 340. The ‘CH3 domain’ of a human IgG Fc region usually extends from about amino acid 341 to about amino acid residue 447 of a human IgG (i.e. comprises the residues C-terminal to a CH2 domain). In embodiments of the present invention, the variant IgG Fc region may be selected from IgG1, IgG2, IgG3 or IgG4, preferably the IgG Fc region of IgG1. IgG1 Fc may be written in the alternative as Fcγ1. A ‘hinge region’ is generally defined as stretching from Glu 216 to Pro 230 of human IgG1 (Burton, (1985) Molec. Immunol. 22: 161-206). Hinge regions of other IgG isotypes may be aligned with the IgG1 sequence by placing the first and last cysteine residues forming inter-heavy chain S—S bonds in the same positions.

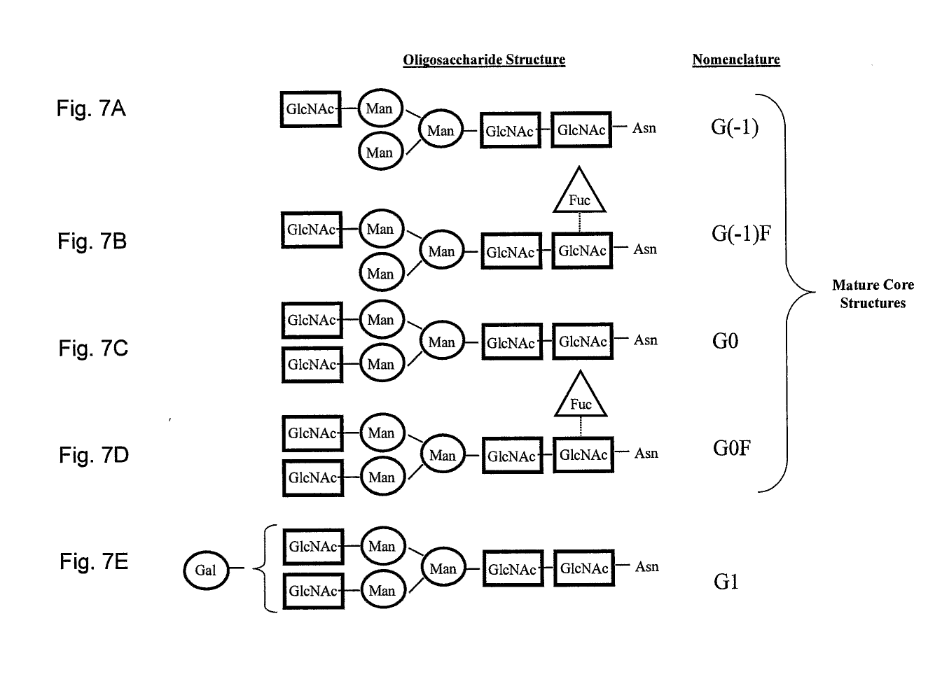

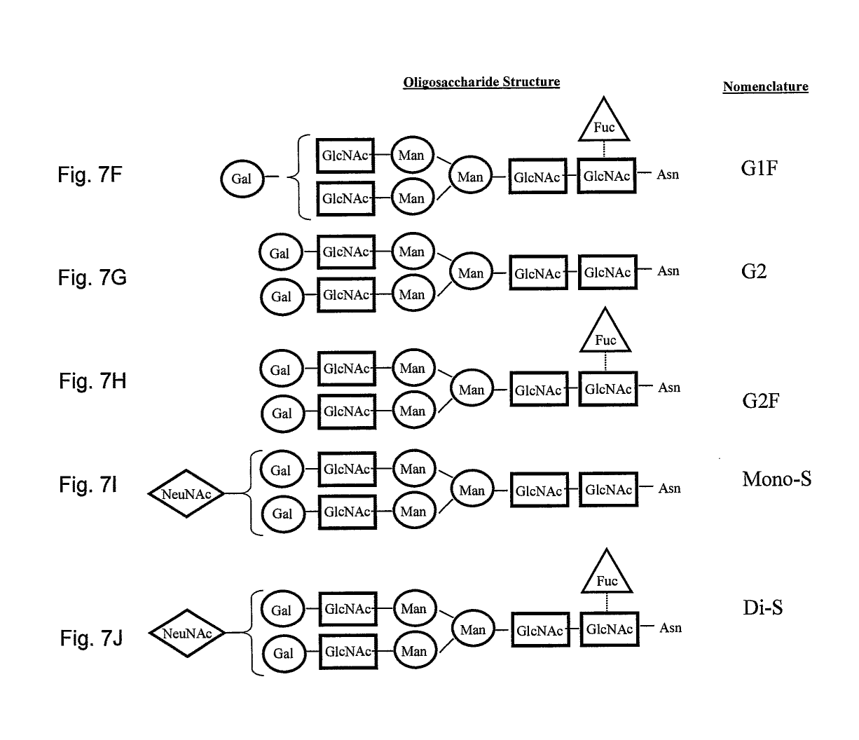

All native antibodies contain carbohydrate at conserved positions in the constant region of the heavy chain. Each antibody isotype has a distinct variety of N-linked carbohydrate structures. The Fc region of IgG1 has a single N-linked biantennary carbohydrate at Asn297 of the CH2 domain. The fully processed (mature) carbohydrate structure attached to Asn297 is depicted in FIG. 7.

A functional Fc region possesses an effector function of a native sequence Fc region for example: C1q binding, CDC, Fc receptor binding, ADCC, phagocytosis, down regulation of cell surface receptors (e.g. B cell receptor), etc. Such effector functions generally require the Fc region to be combined with a binding domain (e.g. an antibody variable domain) and can be assessed using various assays as herein disclosed. For example, effector function may be determined by comparing ability of selected IgG Fc polypeptide variants with that of the parent IgG Fc polypeptide in an ADCC assay.

An Fc polypeptide variant with ‘improved effector function’ compared with a parent Fc polypeptide is one which in vitro or in vivo is substantially more effective at mediating ADCC or CDC, when the amounts of polypeptide Fc variant and parent Fc polypeptide used in the assay are essentially the same. Generally, the variants which mediate ADCC more effectively will be identified using the in vitro ADCC assay as herein disclosed, but other assays or methods for determining ADCC activity, e.g. in an animal model etc, are contemplated. The preferred variant is from about 1.01 fold to about 100 fold, e.g. from about 1.10 fold to about 50 fold, more effective at mediating ADCC than the parent, e.g. in the in vitro assay disclosed herein in Example 8.

The term “glycosylation” means the attachment of oligosaccharides (carbohydrates containing two or more simple sugars linked together e.g. from two to about twelve simple sugars linked together) to a glycoprotein. The oligosaccharide side chains are typically linked to the backbone of the glycoprotein through either N- or O-linkages. The oligosaccharides of the present invention occur generally are attached to a CH2 domain of an Fc region as N-linked oligosaccharides.

The carbohydrate moieties of the present invention will be described with reference to commonly used nomenclature for the description of oligosaccharides. A review of carbohydrate chemistry which uses this nomenclature is found in Hubbard et al. Ann. Rev. Biochem. 50:555-583 (1981). This nomenclature includes, for instance, Man, which represents mannose; GlcNAc, which represents 2-N-acetylglucosamine; Gal which represents galactose; Fuc for fucose; and Glc, which represents glucose. Sialic acids are described by the shorthand notation NeuNAc, for 5-N-acetylneuraminic acid, and NeuNGc for 5-glycolylneuraminic. The abbreviations for the structures are further detailed in FIG. 7.

“N-linked glycosylation” refers to the attachment of the carbohydrate moiety to an asparagine residue in a glycoprotein chain. The skilled artisan will recognize that, for example, each of murine IgG1, IgG2a, IgG2b and IgG3 as well as human IgG1, IgG2, IgG3, IgG4, IgA and IgD CH2 domains have a single site for N-linked glycosylation. IgG has a single site at amino acid residue 297 (Kabat et al. Sequences of Proteins of Immunological Interest, 1991).

For the purposes herein, a “mature core carbohydrate structure” refers to a processed core carbohydrate structure attached to an Fc region which generally consists of the following carbohydrate structure GlcNAc-GlcNAc-Man-(Man-GlcNAc)₂ typical of biantennary oligosaccharides represented schematically below.

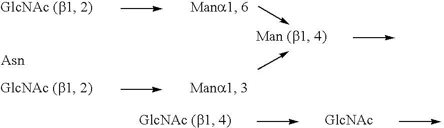

This term specifically includes G(−1) forms of the core mature core carbohydrate structure lacking a β1,2 GlcNAc residue (see, FIGS. 7A and B). Generally however, the core carbohydrate structure includes both β1,2 GlcNAc residues (see, FIGS. 7C and D). The mature core carbohydrate structure herein generally is not hypermannosylated. The mature core may also include fucose in a β1,6 linkage to the GlcNAc at the reducing end of the sugar (see, FIG. 7A-7D).

It will be understood by one of skill in the art that the N-linked oligosaccharide structures attached to an Fc polypeptide may comprise a mature core carbohydrate that further incorporates additional carbohydrate moieties. For example the core structure may further comprise a bisecting GlcNAc sugar moieties and/or terminal Gal, NeuNAc or Man sugar moieties (for example see, FIG. 7E-7J).

A “bisecting GlcNAc” is a GlcNAc residue attached to the β1,4 mannose of the mature core carbohydrate structure. The bisecting GlcNAc can be enzymatically attached to the mature core carbohydrate structure by a β(1,4)-N-acetylglucosaminyltransferase III enzyme (GnTIII). Certain cell types (e.g., CHO cells) do not normally express GnTIII (Stanley et al. J. Biol. Chem. 261:13370-13378 (1984)), but may be engineered to do so (Umana et al. Nature Biotech. 17:176-180 (1999)).

### DETAILED DESCRIPTION

A ribosome translation system employed in a method of the invention may be prokaryotic or eukaryotic. Both are established in the art for display and selection of a number of different binding molecules. See for example: Mattheakis et al., (1994) PNAS USA 91: 9022-9026; Mattheakis et al., (1996) Methods Enzymol. 267: 195-207; Gersuk et al., (1997) Biotech. and Biophys. Res. Com. 232: 578-582; Hanes and Pluckthun (1997) PNAS USA 94: 4937-4942; Hanes et al., (1998) PNAS USA 95: 14130-50; He and Taussig (1997) Nucleic Acids Res. 25: 5132-5234; Hanes et al. (2000) Meth. Enzymol. 328: 403-430 and Plückthun et al. (2000) Adv. Prot. Chem. 55: 367-403).

A construct for ribosome display may comprise a RNA polymerase promoter (e.g. T7 polymerase promoter), ribosome binding site, Kozak consensus sequence, initiation codon and coding sequence of polypeptide, peptide or protein. One or more nucleotide sequences encoding one or more detection tags may be included to provide for production of a polypeptide, peptide or protein further comprising one or more detection tags (e.g. histidine tag). One or more additional features may incorporated into a construct for use in ribosome display, e.g. as disclosed in WO01/75097.

An mRNA translation system used in an embodiment of the invention may be any suitable available system. A prokaryotic or eukaryotic translation system may be used, for example crude E. coli or wheat (e.g. as supplied by Roche, Invitrogen) lysate, rabbit reticulocyte lysate (e.g. as supplied by Ambion, Promega) or a reconstituted system such as PURE (reported by Shimizu et al., (2001) Nat. Biotechnol. 19: 751-755).

In some embodiments of the present invention, mRNA molecules for incubation in the translation system are provided by means of RT-PCR reactions in which at least one of the RT-PCR primers is a mutagenic primer encoding a diversity of different sequences for inclusion in a defined region of the mRNA coding region. For example, a defined region may be one encoding a CDR of an antibody molecule, including but not limited to a, CDR3 of an antibody VH domain.

A defined region for mutation may comprise a residue found necessary for overall protein stability (Proba et al., (1998) J. Mol. Biol. 275: 245-253) or be an area of a protein which is likely to be involved in early aggregation events, such as exposed loops. A defined region for maturation may also comprise residues, that effect function, that are required for binding/contacting other protein molecules, but may also comprise other residues that may influence said contact sites. For example, a defined region may be encoding a hinge and/or CH2 and/or CH3 domain of an Fc molecule. In one embodiment, a defined region encodes at least a portion of a CH2 domain. In another embodiment, a defined region encodes at least a portion of a CH3 domain. In still another embodiment, a defined region encodes at least a portion of a CH2 and a portion of a CH3 region.

Following selection and retrieval of nucleic acid encoding the displayed Fc polypeptide variant, the nucleic acid may be used in provision of the encoded Fc polypeptide variant or may be used in provision of further nucleic acid (e.g. by means of an amplification reaction such as PCR). Selected mRNA may be subjected to RT-PCR to generate cDNA copies. Nucleic acid encoding component parts of an Fc polypeptide variant may be used in provision of further molecules, for instance reformatted antibody molecules, fusion proteins, immunoadhesins and so on. Thus, for example, nucleic acid encoding the VH and VL domains of a selected scFv antibody molecule may be used in construction of sequences encoding antibody molecules of other formats such as Fab molecules or whole antibody. Alternatively, nucleic acid encoding the CH2 and/or CH3 domains of an Fc molecule may be used in construction of sequences encoding antibody molecules of other formats such as dimeric Fcs, fusion proteins, immunoadhesions, whole antibodies, and so on.

In a method of the invention, DNA encoding the selected Fc polypeptide variant or a polypeptide chain of the selected Fc polypeptide variant may be mutated to encode a polypeptide that comprises an amino acid sequence that differs from the selected Fc polypeptide variant or polypeptide chain of the selected Fc polypeptide variant. Mutated DNA encoding said polypeptide may be provided in an expression system for production of a product, which product is said polypeptide. A method may further comprise isolating or purifying the product, optionally formulating the product into a composition comprising at least one additional component.

Furthermore, nucleic acid may be subject to any technique available in the art for alteration or mutation of its sequence. This may be used to provide a derivative sequence. A sequence may be provided which encodes a derivative of the selected Fc polypeptide variant or component thereof, for example a derivative that comprises an amino acid sequence that differs from the selected Fc polypeptide variant or component thereof by addition, deletion, insertion and/or substitution of one or more amino acid sequences. A method providing such a derivative may provide a fusion protein or conjugate wherein an additional peptide or polypeptide moiety is joined to the Fc polypeptide variant or component thereof, e.g. a toxin or label.

Encoding nucleic acid, whether reformatted or not, may be used in production of the encoded polypeptide or peptide using any technique available in the art for provision of polypeptides and peptides by recombinant expression.

An amino acid alteration refers to a change in the amino acid sequence of a predetermined amino acid sequence by addition, deletion, substitution and/or insertion of an amino acid residue. Alteration may comprise replacing one or more amino acid residue with a non-naturally occurring or non-standard amino acid, modifying one or more amino acid residue into a non-naturally occurring or non-standard form, or inserting one or more non-naturally occurring or non-standard amino acid into the sequence. The preferred amino acid alteration herein is a substitution. Preferred numbers and locations of alterations in sequences of embodiments of the invention are described elsewhere herein. Naturally occurring amino acids include the 20 ‘standard’ L-amino acids identified as alanine (Ala), arginine (Arg), asparagine (Asn), aspartic acid (Asp), cysteine (Cys), glutamine (Gln), glutamic acid (Glu), glycine (Gly), histidine (His), Isoleucine (Ile), leucine (Leu), lysine (Lys), methionine (Met), phenylalanine (Phe), proline (Pro), serine (Ser), threonine (Thr), tryptophan (Trp), tyrosine (Tyr), and valine (Val). Non-standard amino acids include any other residue that may be incorporated into a polypeptide backbone or result from modification of an existing amino acid residue. Non-standard amino acids may be naturally occurring or non-naturally occurring. Several naturally occurring non-standard amino acids are known in the art, such as 4-hydroxyproline, 5-hydroxylysine, 3-methylhistidine, N-acetylserine, etc. (Voet & Voet, 1995). Those amino acid residues that are derivatized at their N-alpha position will only be located at the N-terminus of an amino-acid sequence. Normally, in embodiments of the present invention an amino acid is an L-amino acid, but in some embodiments it may be a D-amino acid. Alteration may therefore comprise modifying an L-amino acid into, or replacing it with, a D-amino acid. Methylated, acetylated and/or phosphorylated forms of amino acids are also known, and amino acids in the present invention may be subject to such modification.

Amino acid sequences in Fc polypeptide variants of embodiments of the invention may comprise non-natural or non-standard amino acids as described above. In some embodiments non-standard amino acids (e.g. D-amino acids) may be incorporated into an amino acid sequence during synthesis, while in other embodiments the non-standard amino acids may be introduced by modification or replacement of the ‘original’ standard amino acids after synthesis of the amino acid sequence.

The term ‘antibody’ describes an immunoglobulin whether natural or partly or wholly synthetically produced. It covers monoclonal antibodies (including full length monoclonal antibodies), polyclonal antibodies, multispecific antibodies (e.g. bispecific antibodies), and antibody fragments, so long as they exhibit the desired biological activity. The term also covers any polypeptide or protein comprising an antibody antigen-binding site. It must be understood here that embodiments of the invention do not relate to the antibodies in natural form, that is to say they are not in their natural environment but that they have been able to be isolated or obtained by purification from natural sources, or else obtained by genetic recombination, or by chemical synthesis, and that they can then contain unnatural amino acids as will be described later. Antibody fragments that comprise an antibody antigen-binding site include, but are not limited to molecules such as Fab, Fab′, Fab′-SH, scFv, Fv, dAb, Fd and diabodies.

As antibodies can be modified in a number of ways, the term ‘antibody’ should be construed as covering any binding member or substance having an antibody antigen-binding site with the required specificity and/or binding to antigen. Thus, this term covers antibody fragments and derivatives, including any polypeptide comprising an antibody antigen-binding site, whether natural or wholly or partially synthetic. Chimeric molecules comprising an antibody antigen-binding site, or equivalent, fused to another polypeptide (e.g. derived from another species or belonging to another antibody class or subclass) are therefore included. Cloning and expression of chimeric antibodies are described in EP120694 and EP125023, and a large body of subsequent literature.

Further techniques available in the art of antibody engineering have made it possible to isolate human and humanised antibodies. For example, human hybridomas can be made as described by Kontermann & Dubel (2001, Antibody Engineering, Springer). Phage display, another established technique for generating binding members has been described in detail in many publications such as Kontermann & Dubel (2001; supra) and WO92/01047. Transgenic mice in which the mouse antibody genes are inactivated and functionally replaced with human antibody genes while leaving intact other components of the mouse immune system, can be used for isolating human antibodies (Mendez et al., (1997) Nature Genet. 15(2): 146-56). For example, the VelociGene® technology from Regeneron replaces only the variable regions of mouse immune loci (heavy chain V, D, and J segments, and light chain V and J segments) with corresponding human variable sequences in situ, whilst leaving the normal mouse constant regions intact. Synthetic antibody molecules may be created by expression from genes generated by means of oligonucleotides synthesized and assembled within suitable expression vectors, for example as described by Knappik et al. (2000, J. Mol. Biol. 296(1): 57-86) or Krebs et al. (2001, J. Immunol. Methods 254: 67-84).

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward et al., (1989) Nature 341: 484-5; McCafferty et al., (1990) Nature 348:552-4; Holt et al., (2003) Trends Biotechnol. 21: 484-90), which consists of a VH or a VL domain; (v) isolated CDR regions, which may be displayed on a scaffold; (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., (1988) Science 242: 423-426; Huston et al., (1988) PNAS USA 85: 5879-83); (viii) bispecific single chain Fv dimers (WO93/011161) and (ix) ‘diabodies’, multivalent or multispecific fragments constructed by gene fusion (WO94/13804; Holliger et al., (1993) PNAS USA 90: 6444-8). Fv, scFv or diabody molecules may be stabilized by the incorporation of disulphide bridges linking the VH and VL domains (Reiter et al., (1996) Nature Biotech. 14: 1239-45). Minibodies comprising a scFv joined to a CH3 domain may also be made (Hu et al., (1996) Cancer Res. 56: 3055-61). Other examples of binding fragments are Fab′, which differs from Fab fragments by the addition of a few residues at the carboxyl terminus of the heavy chain CH1 domain, including one or more cysteines from the antibody hinge region, and Fab′-SH, which is a Fab′ fragment in which the cysteine residue(s) of the constant domains bear a free thiol group.

It will be understood by one of skill in the art that one or more of the antibody fragments disclosed herein may be joined to a selected Fc polypeptide variant or a polypeptide chain of the selected Fc polypeptide variant to generate a fusion and/or conjugated protein.

In a following provision in accordance with a method of the invention, a Fc polypeptide variant may be isolated and/or purified (e.g. using an antibody) for instance after production by expression from encoding nucleic acid (for which see below). Thus, a Fc polypeptide variant may be provided free or substantially free from contaminants. A Fc polypeptide variant may be provided free or substantially free of other polypeptides. The isolated and/or purified Fc polypeptide variant may be used in formulation of a composition, which may include at least one additional component, for example a pharmaceutical composition including a pharmaceutically acceptable excipient, vehicle or carrier. A composition including a polypeptide according to an embodiment of the invention may be used in prophylactic and/or therapeutic treatment as discussed below.

It is also specifically contemplated that an Fc polypeptide variant of the invention may contain inter alia one or more additional amino acid residue substitutions, mutations and/or modifications which result in an antibody with preferred characteristics including but not limited to: increased serum half life, increase binding affinity, reduced immunogenicity, increased production, enhanced or reduced ADCC or CDC activity, altered glycosylation and/or disulfide bonds and modified binding specificity.

An Fc polypeptide variant of the present invention may be combined with other Fc modifications, including but not limited to modifications that alter effector function. The invention encompasses combining an Fc variant of the invention with other Fc modifications to provide additive, synergistic, or novel properties in antibodies or Fc fusions. Such modifications may be in the CH1, CH2, or CH3 domains or a combination thereof. It is contemplated that an Fc polypeptide variant of the invention will enhance the property of the modification with which it is combined. For example, if an Fc polypeptide variant of the invention is combined with a mutant known to bind FcγRIIIA with a higher affinity than a molecule comprising a wild type Fc region; the combination with a mutant of the invention results in a greater fold enhancement in FcγRIIIA affinity.

In one embodiment, an Fc polypeptide variant of the present invention may be combined with other known Fc mutations such as those disclosed in Duncan et al, 1988, Nature 332:563-564; Lund et al., 1991, J. Immunol 147:2657-2662; Lund et al, 1992, Mol Immunol 29:53-59; Alegre et al, 1994, Transplantation 57:1537-1543; Hutchins et al., 1995, Proc Natl. Acad Sci USA 92:11980-11984; Jefferis et al, 1995, Immunol Lett. 44:111-117; Lund et al., 1995, Faseb J 9:115-119; Jefferis et al, 1996, Immunol Lett 54:101-104; Lund et al, 1996, Immunol 157:4963-4969; Armour et al., 1999, Eur J Immunol 29:2613-2624; Idusogie et al, 2000, J Immunol 164:4178-4184; Reddy et al, 2000, J Immunol 164:1925-1933; Xu et al., 2000, Cell Immunol 200:16-26; Idusogie et al, 2001, J Immunol 166:2571-2575; Shields et al., 2001, J Biol Chem 276:6591-6604; Jefferis et al, 2002, Immunol Lett 82:57-65; Presta et al., 2002, Biochem Soc Trans 30:487-490); U.S. Pat. Nos. 5,624,821; 5,885,573; 6,194,551; U.S. Patent Application Nos. 60/601,634 and 60/608,852; PCT Publication Nos. WO 00/42072 and WO 99/58572.

It has been demonstrated that glycosylation of IgG is crucial for regulation of cytotoxicity and anti-inflammatory potential of IgG. It particular the absence of fucose correlates with improved ADCC activity (Niwa et al. (2004) Cancer Res. 64: 2127-2133)). Similarly, presence of a bisecting N-acetylglucosamine (GlcNAc) has also been shown to increase ADCC activity (Umana et al. (1999), Nat. Biotechnol 17:176-180). In addition, the presence of sialic acid correlates_with improved anti-inflammatory activity of IgG (Kaneko, et al., (2006) Science 313: 670-3). Accordingly, instant invention provides a novel method for increasing the percentage of Fc polypeptides comprising a mature core carbohydrate structure which lacks fucose (see FIGS. 7A, C, E, G and I) and/or has sialic acid present in a composition (see FIGS. 7I and J).

In one embodiment, the invention provides a method of increasing the percentage of Fc polypeptides comprising a mature core carbohydrate structure which lacks fucose present in a composition, said method comprising:
(a) introducing at least one mutation into a nucleic acid encoding the Fc polypeptide, wherein the mutation results in a substitution at amino acid residue 243; and
(b) expressing the mutated nucleic acid in mammalian cells to produce a glycosylated composition of Fc polypeptides,
   wherein the numbering of the residues is that of the EU index as in Kabat.

In certain embodiments, the percentage of Fc polypeptides comprising a mature core carbohydrate structure which lacks fucose present in the composition is increased to between about 20% to about 50%, or between about 20% to about 40%, or between about 20% to about 30%, or between 30% to about 50%, or between about 30% to about 40%. In a specific embodiment, the percentage of Fc polypeptides comprising a mature core carbohydrate structure which lacks fucose present in the composition is increased to between about 30% and about 40%. In other embodiments, the percentage of Fc polypeptides comprising a mature core carbohydrate structure which lacks fucose present in the composition is increased to at least about 20%, or at least about 25%, or at least about 30%, or at least about 35%, or at least about 40%, or at least about 45%, or at least about 50%.

In another embodiment, the method will also increase the percentage of Fc polypeptides comprising a mature core carbohydrate structure which has sialic acid.

In certain embodiments, the percentage of Fc polypeptides comprising a mature core carbohydrate structure which has sialic acid is increased to between about 5% to about 40%, or between about 5% to about 30%, or between about 5% to about 20%, or between about 10% to about 40%, or between about 10% to about 30%, or between about 10% to about 20%. In other embodiments, the percentage of Fc polypeptides comprising a mature core carbohydrate structure which has sialic acid is increased to at least about 5%, or at least about 10%, or at least about 15%, or at least about 20%, or at least about 25%, or at least about 30%, or at least about 35%, or at least about 40%.

In another embodiment, the method will also increase the percentage of Fc polypeptides comprising a mature core carbohydrate structure which has a bisecting GlcNAc.

In certain embodiments, the percentage of Fc polypeptides comprising a mature core carbohydrate structure which has a bisecting GlcNAc is increased to between about 5% to about 30%, or between about 5% to about 20%, or between about 5% to about 10%. In other embodiments, the percentage of Fc polypeptides comprising a mature core carbohydrate structure which has a bisecting GlcNAc is increased to at least about 5%, or at least about 10%, or at least about 15%, or at least about 20%, or at least about 25%, or at least about 30%.

In specific embodiments the mutation results in a substitution at position 243 selected from the group consisting of 243L, 243G, 243H and 243I.

It will be understood by one of skill in the art that any animal cell which is capable of producing a glycoprotein comprising a mature core carbohydrate may be used in the methods of the instant invention. Such animal host cells include, but are not limited to, CEK, CHO, VERY, BHK, Hela, COS, MDCK, 293, 3T3, WI38, NS0, and in particular, neuronal cell lines such as, for example, SK-N-AS, SK-N-FI, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl. Acad. Sci. USA, 1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Viral. Methods 48: 211-221), G355-5, PG-4 Cat normal astrocyte (Haapala et al., 1985, J. Viral. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst.

In other embodiments, an Fc polypeptide variant of the present invention comprises one or more engineered glycoforms, i.e., a carbohydrate composition that is covalently attached to a molecule comprising an Fc region. Engineered glycoforms may be useful for a variety of purposes, including but not limited to enhancing or reducing effector function. Engineered glycoforms may be generated by any method known to one skilled in the art, for example by using engineered or variant expression strains, by co-expression with one or more enzymes, for example β(1,4)-N-acetylglucosaminyltransferase III (GnTI11), by expressing a molecule comprising an Fc region in various organisms or cell lines from various organisms, or by modifying carbohydrate(s) after the molecule comprising Fc region has been expressed. Methods for generating engineered glycoforms are known in the art, and include but are not limited to those described in Umana et al, 1999, Nat. Biotechnol 17:176-180; Davies et al., 20017 Biotechnol Bioeng 74:288-294; Shields et al, 2002, J Biol Chem 277:26733-26740; Shinkawa et al., 2003, J Biol Chem 278:3466-3473) U.S. Pat. No. 6,602,684; U.S. Ser. No. 10/277,370; U.S. Ser. No. 10/113,929; PCT WO 00/61739A1; PCT WO 01/292246A1; PCT WO 02/311140A1; PCT WO 02/30954A1; Potillegent™ technology (Biowa, Inc. Princeton, N.J.); GlycoMAb™ glycosylation engineering technology (GLYCART biotechnology AG, Zurich, Switzerland). See, e.g., WO 00061739; EA01229125; US 20030115614; Okazaki et al., 2004, JMB, 336: 1239-49.

A convenient way of producing an Fc polypeptide variant according to an embodiment of the present invention is to express it from the nucleic acid encoding it, by use of the nucleic acid in an expression system. Accordingly, an embodiment of the present invention also encompasses a method of making an Fc polypeptide variant (as disclosed), the method including expression from nucleic acid encoding the polypeptide (generally nucleic acid according to an embodiment of the invention). This may conveniently be achieved by growing a host cell in culture, containing such a vector, under appropriate conditions which cause or allow expression of the polypeptide. Fc polypeptide variants may also be expressed in in vitro systems, such as reticulocyte lysate.

Systems for cloning and expression of a polypeptide in a variety of different host cells are well known. Suitable host cells include bacteria, eukaryotic cells such as mammalian and yeast, and baculovirus systems. Mammalian cell lines available in the art for expression of a heterologous polypeptide include Chinese hamster ovary (CHO) cells, HeLa cells, baby hamster kidney cells, COS cells and many others. A common, preferred bacterial host is E. coli. Suitable vectors can be chosen or constructed, containing appropriate regulatory sequences, including promoter sequences, terminator fragments, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Ausubel et al. eds., John Wiley & Sons, 2006.

Nucleic acid encoding an Fc polypeptide variant may be provided in accordance with methods of the invention.

Generally, nucleic acid according to an embodiment of the present invention is provided as an isolate, in isolated and/or purified form, or free or substantially free of contaminants. Nucleic acid may be wholly or partially synthetic and may include genomic DNA, cDNA or RNA.

Nucleic acid may be provided as part of a replicable vector, and also provided by embodiments of the present invention is a vector including nucleic acid encoding an Fc polypeptide variant of the invention, particularly any expression vector from which the encoded polypeptide can be expressed under appropriate conditions, and a host cell containing any such vector or nucleic acid. An expression vector in this context is a nucleic acid molecule including nucleic acid encoding a polypeptide of interest and appropriate regulatory sequences for expression of the polypeptide, in an in vitro expression system, e.g. reticulocyte lysate, or in vivo, e.g. in eukaryotic cells such as COS or CHO cells or in prokaryotic cells such as E. coli.

A host cell may be provided containing nucleic acid as disclosed herein. The nucleic acid may be integrated into the genome (e.g. chromosome) of the host cell. Integration may be promoted by inclusion of sequences which promote recombination with the genome, in accordance with standard techniques. The nucleic acid may be on an extra-chromosomal vector within the cell.

The nucleic acid may be introduced into a host cell. The introduction, which may (particularly for in vitro introduction) be generally referred to without limitation as ‘transformation’ or ‘transfection’, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage.

Marker genes such as antibiotic resistance or sensitivity genes may be used in identifying clones containing nucleic acid of interest, as is well known in the art.

The introduction may be followed by causing or allowing expression from the nucleic acid, e.g. by culturing host cells (which may include cells actually transformed although more likely the cells will be descendants of the transformed cells) under conditions for expression of the gene, so that the encoded polypeptide is produced. If the Fc polypeptide variant is expressed coupled to an appropriate signal leader peptide it may be secreted from the cell into the culture medium. Following production by expression, the polypeptide may be isolated and/or purified from the host cell and/or culture medium, as the case may be, and subsequently used as desired, e.g. in the formulation of a composition which may include one or more additional components, such as a pharmaceutical composition which includes one or more pharmaceutically acceptable excipients, vehicles or carriers (e.g. see below).

Following production of an Fc polypeptide variant by expression, its activity, e.g. ability to bind receptor or ligand or other specific binding pair member, can be tested by a number of methods as detailed below and in the present Examples. Briefly, a radio immunoassay (RIA) can be used to determine an improvement in binding of an Fc polypeptide variant to an Fc ligand (e.g. FcγRIIIa or C1q) or a reduction in binding to an Fc ligand (e.g. FcγRIIb) compared to that of the wild-type Fc polypeptide. A further test makes use of an AlphaScreen™ assay (Perkin Elmer), which is a bead based non-radioactive assay. A biological interaction brings Donor and Acceptor beads together resulting in a cascade of chemical reactions that acts to produce a greatly amplified signal with output in the 520-620 nm range. In addition, an ELISA can be used to assess binding of Fc polypeptide variants to an Fc ligand (e.g. FcγRIIb, FcγRIIIa or C1q).

In certain embodiments, an Fc polypeptide variant of the embodiments has improved binding affinity for an FcR, as compared to the parent Fc polypeptide. In some embodiments, the binding affinity of an Fc polypeptide variant to FcR is improved by about 1.10 fold to about 100 fold, or about 1.15 fold to about 50 fold, or about 1.20 fold to about 25 fold, as compared to the parent Fc polypeptide, where FcR binding affinity is determined (e.g. as disclosed in the Examples herein). In other embodiments, the binding affinity of an Fc polypeptide variant to FcR is improved by at least about 1.10 fold, or at least about 1.20 fold, or at least about 1.30 fold, or at least about 1.4 fold, or at least about 1.5 fold, or at least about 1.6 fold, or at least about 1.70 fold, or at least about 1.8 fold, or at least about 1.9 fold, or at least about 2.0 fold, or at least about 2.5 fold, or at least about 3 fold, or at least about 3.5 fold, or at least about 4.0 fold, or at least about 4.5 fold, or at least about 5.0 fold, or at least about 5.5 fold, or at least about 6 fold, or at least about 7 fold, or at least about 8 fold, or at least about 10 fold, as compared to the parent Fc polypeptide, where FcR binding affinity is determined (e.g. as disclosed in the Examples herein). In one embodiment, the FcR that the Fc polypeptide variant has improved binding to is FcγRIIIa. In another embodiment, the FcR that the Fc polypeptide variant has improved binding to is FcγRIIb. In still another embodiment, the FcR that the Fc polypeptide variant has improved binding to is FcγRIIa. In a specific embodiment, the FcR that the Fc polypeptide variant has improved binding to is FcγRIIIa F158. In another specific embodiment, the FcR that the Fc polypeptide variant has improved binding to is FcγRIIIa V158.

In other embodiments, an Fc polypeptide variant of the embodiments has reduced binding affinity for an FcR, as compared to the parent Fc polypeptide. In some embodiments, the binding affinity of an Fc polypeptide variant to FcR is reduced by about 1.10 fold to about 100 fold, or about 1.15 fold to about 50 fold, or about 1.20 fold to about 25 fold, as compared to the parent Fc polypeptide, where FcR binding affinity is determined (e.g. as disclosed in the Examples herein). In other embodiments, the binding affinity of an Fc polypeptide variant to FcR is reduced by at least about 1.10 fold, or at least about 1.20 fold, or at least about 1.30 fold, or at least about 1.4 fold, or at least about 1.5 fold, or at least about 1.6 fold, or at least about 1.70 fold, or at least about 1.8 fold, or at least about 1.9 fold, or at least about 2.0 fold, or at least about 2.5 fold, or at least about 3 fold, or at least about 3.5 fold, or at least about 4.0 fold, or at least about 4.5 fold, or at least about 5.0 fold, or at least about 5.5 fold, or at least about 6 fold, or at least about 7 fold, or at least about 8 fold, or at least about 10 fold, as compared to the parent Fc polypeptide, where FcR binding affinity is determined (e.g. as disclosed in the Examples herein). In one embodiment, the FcR that the Fc polypeptide variant has reduced binding to is FcγRIIIa. In another embodiment, the FcR that the Fc polypeptide variant has reduced binding to is FcγRIIb. In still another embodiment, the FcR that the Fc polypeptide variant has reduced binding to is FcγRIIa. In a specific embodiment, the FcR that the Fc polypeptide variant has reduced binding to is FcγRIIIa F158. In another specific embodiment, the FcR that the Fc polypeptide variant has reduced binding to is FcγRIIIa V158.

In certain embodiment, an Fc polypeptide variant of the embodiments has improved binding affinity for C1q, as compared to the parent Fc polypeptide. In a specific embodiment, the binding affinity of an Fc polypeptide variant to C1q is improved by about 1.10 fold to about 100 fold, or about 1.15 fold to about 50 fold, or about 1.20 fold to about 25 fold, as compared to the parent Fc polypeptide, where C1q binding affinity is determined (e.g. as disclosed in the Examples herein). In other embodiments, the binding affinity of an Fc polypeptide variant to C1q is improved by at least about 1.10 fold, or at least about 1.20 fold, or at least about 1.30 fold, or at least about 1.4 fold, or at least about 1.5 fold, or at least about 1.6 fold, or at least about 1.70 fold, or at least about 1.8 fold, or at least about 1.9 fold, or at least about 2.0 fold, or at least about 2.5 fold, or at least about 3 fold, or at least about 3.5 fold, or at least about 4.0 fold, or at least about 4.5 fold, or at least about 5.0 fold, or at least about 5.5 fold, or at least about 6 fold, or at least about 7 fold, or at least about 8 fold, or at least about 10 fold, as compared to the parent Fc polypeptide, where C1q binding affinity is determined (e.g. as disclosed in the Examples herein).

In other embodiments, an Fc polypeptide variant of the embodiments has reduced binding affinity for C1q, as compared to the parent Fc polypeptide. In a specific embodiment, the binding affinity of an Fc polypeptide variant to C1q is reduced by about 1.10 fold to about 100 fold, or about 1.15 fold to about 50 fold, or about 1.20 fold to about 25 fold, as compared to the parent Fc polypeptide, where C1q binding affinity is determined (e.g. as disclosed in the Examples herein). In other embodiments, the binding affinity of an Fc polypeptide variant to C1q is reduced by at least about 1.10 fold, or at least about 1.20 fold, or at least about 1.30 fold, or at least about 1.4 fold, or at least about 1.5 fold, or at least about 1.6 fold, or at least about 1.70 fold, or at least about 1.8 fold, or at least about 1.9 fold, or at least about 2.0 fold, or at least about 2.5 fold, or at least about 3 fold, or at least about 3.5 fold, or at least about 4.0 fold, or at least about 4.5 fold, or at least about 5.0 fold, or at least about 5.5 fold, or at least about 6 fold, or at least about 7 fold, or at least about 8 fold, or at least about 10 fold, as compared to the parent Fc polypeptide, where C1q binding affinity is determined (e.g. as disclosed in the Examples herein).

Fc polypeptide variants may also be assayed for their cellular activity, e.g., ability to mediate ADCC or CDC activity. To assess the ability of any particular antibody to mediate lysis of the target cell by ADCC, an antibody of interest is added to target cells in combination with immune effector cells, which may be activated by the antigen antibody complexes resulting in cytolysis of the target cell. Cytolysis is generally detected by the release of label (e.g. radioactive substrates, fluorescent dyes or natural intracellular proteins) from the lysed cells. Useful effector cells for such assays include peripheral blood mononuclear cells (PBMC) and Natural Killer (NK) cells. Specific examples of in vitro ADCC assays are described in Wisecarver et al., 1985, 79:277; Bruggemann et al., 1987, J Exp Med 166:1351; Wilkinson et al., 2001, J Immunol Methods 258:183; Patel et al., 1995 J Immunol Methods 184:29 and as described herein (see Examples). Alternatively, or additionally, ADCC activity may be assessed in vivo, e.g., in an animal model such as that disclosed in Clynes et al., 1998, PNAS USA 95:652. To assess complement activation, a CDC assay, e.g. as described in Gazzano-Santoro et al., 1996, J. Immunol. Methods, 202:163, may be performed.

In certain embodiments, an Fc polypeptide variant of the embodiments has improved ADCC activity, as compared to the parent Fc polypeptide. In some embodiments, ADCC activity improved by about 1.10 fold to about 100 fold, or about 1.15 fold to about 50 fold, or about 1.20 fold to about 25 fold, as compared to the parent Fc polypeptide, where ADCC activity is determined, (e.g. as disclosed in the Examples herein). In other embodiments, the ADCC activity of an Fc polypeptide variant to FcR is improved by at least about 1.10 fold, 1.10 fold, or at least about 1.20 fold, or at least about 1.30 fold, or at least about 1.4 fold, or at least about 1.5 fold, or at least about 1.6 fold, or at least about 1.70 fold, or at least about 1.8 fold, or at least about 1.9 fold, or at least about 2.0 fold, or at least about 2.5 fold, or at least about 3 fold, or at least about 3.5 fold, or at least about 4.0 fold, or at least about 4.5 fold, or at least about 5.0 fold, or at least about 5.5 fold, or at least about 6 fold, or at least about 7 fold, or at least about 8 fold, or at least about 10 fold, or at least about 25 fold, as compared to the parent Fc polypeptide, where ADCC activity is determined (e.g. as disclosed in the Examples herein).

In other embodiments, an Fc polypeptide variant of the embodiments has reduced ADCC activity, as compared to the parent Fc polypeptide. In some embodiments, ADCC activity reduced by about 1.10 fold to about 100 fold, or about 1.15 fold to about 50 fold, or about 1.20 fold to about 25 fold, as compared to the parent Fc polypeptide, where ADCC activity is determined, (e.g. as disclosed in the Examples herein). In other embodiments, the ADCC activity of an Fc polypeptide variant to FcR is reduced by at least about 1.10 fold, 1.10 fold, or at least about 1.20 fold, or at least about 1.30 fold, or at least about 1.4 fold, or at least about 1.5 fold, or at least about 1.6 fold, or at least about 1.70 fold, or at least about 1.8 fold, or at least about 1.9 fold, or at least about 2.0 fold, or at least about 2.5 fold, or at least about 3 fold, or at least about 3.5 fold, or at least about 4.0 fold, or at least about 4.5 fold, or at least about 5.0 fold, or at least about 5.5 fold, or at least about 6 fold, or at least about 7 fold, or at least about 8 fold, or at least about 10 fold, or at least about 25 fold, as compared to the parent Fc polypeptide, where ADCC activity is determined (e.g. as disclosed in the Examples herein).

After an Fc polypeptide variant of an embodiment of the invention has been identified or obtained it may be provided in isolated and/or purified form, it may be used as desired, and it may be formulated into a composition comprising at least one additional component, such as a pharmaceutically acceptable excipient or carrier. Nucleic acid encoding the Fc polypeptide variant may be used to produce the variant for subsequent use. As noted, such nucleic acid may, for example, be isolated from a library or diverse population initially provided and from which the Fc polypeptide variant was produced and identified.

An Fc polypeptide variant in accordance with an embodiment of the present invention may be used in methods of diagnosis or treatment of the human or animal body of subjects, preferably human.

Accordingly, further aspects of the invention provide methods of treatment comprising administration of an Fc polypeptide variant as provided, pharmaceutical compositions comprising such an Fc polypeptide variant, and use of such an Fc polypeptide variant in the manufacture of a medicament for administration, for example in a method of making a medicament or pharmaceutical composition comprising formulating the Fc polypeptide variant with a pharmaceutically acceptable excipient.

Such pharmaceutical compositions may comprise an antibody comprising an Fc polypeptide variant or a fusion protein comprising an Fc polypeptide variant, as provided herein.

The antibody may be selected from one which binds to tumour associated antigens. A tumour associated antigen may be selected from the following list: 707-AP (707 alanine proline), AFP (alpha (α)-fetoprotein), AIM-2 (interferon-inducible protein absent in melanoma 2), ART-4 (adenocarcinoma antigen recognized by T cells 4), BAGE (B antigen), Bcr-abl (breakpoint cluster region-Abelson), CAMEL (CTL-recognized antigen on melanoma), CAP-1 (carcino-embryonic antigen peptide-1), CASP-8 (caspase-8), CDC27 (cell-division-cycle 27), CDK4 (cyclin-dependent kinase 4), CEA (carcino-embryonic antigen), CLCA2 (calcium-activated chloride channel-2), CT (cancer/testis (antigen)), Cyp-B (cyclophilin B), DAM (differentiation antigen melanoma), ELF2 (elongation factor 2), Ep-CAM (epithelial cell adhesion molecule), EphA2, 3 (Ephrin type-A receptor 2, 3), ETV6-AML1 (Ets variant gene 6/acute myeloid leukemia 1 gene ETS), FGF-5 (Fibroblast growth factor-5), FN (fibronectin), G250 (glycoprotein 250), GAGE (G antigen), GnT-V (N-acetylglucosaminyltransferase V), Gp100 (glycoprotein 100 kD), HAGE (helicase antigen), HER-2/neu (human epidermal receptor-2/neurological), HLA-A*0201-R170I (arginine (R) to isoleucine (I) exchange at residue 170 of the α-helix of the α2-domain in the HLA-A2 gene), HSP70-2M (heat shock protein 70-2 mutated), HST-2 (human signet ring tumor-2) hTERT (human telomerase reverse transcriptase), iCE (intestinal carboxyl esterase), IL-13Rα2 (interleukin 13 receptor α2 chain), KIAA0205, LAGE (L antigen), LDLR/FUT (low density lipid receptor/GDP-L-fucose: β-D-galactosidase 2-α-L-fucosyltransferase), MAGE (melanoma antigen), MART-1/Melan-A (melanoma antigen recognized by T cells-1/Melanoma antigen A), MART-2 (melanoma Ag recognized by T cells-2), MC1R (melanocortin 1 receptor), M-CSF (macrophage colony-stimulating factor gene), MUC1,2 (mucin 1,2), MUM-1, -2, -3 (melanoma ubiquitous mutated 1,2,3), NA88-A (NA cDNA clone of patient M88), Neo-PAP (Neo-poly(A) polymerase—NPM/ALK), nucleophosmin/anaplastic lymphoma kinase fusion protein), NY-ESO-1 (New York—esophageous 1), OA1 (ocular albinism type 1 protein), OGT (O-linked N-acetylglucosamine transferase gene), OS-9, P15 (protein 15), p190 minor bcr-abl (protein of 190 KD bcr-abl), Pml/RARα (promyelocytic leukemia/retinoic acid receptor α), PRAME (preferentially expressed antigen of melanoma), PSA (prostate-specific antigen), PSMA (prostate-specific membrane antigen) PTPRK (receptor-type protein-tyrosine phosphatase kappa), RAGE (renal antigen), RU1,2 (renal ubiquitous 1,2), SAGE (sarcoma antigen), SART-1, -2, -3 (squamous antigen rejecting tumor 1, 2, 3), SSX-2 (synovial sarcoma, X breakpoint 2), Survivin-2B (intron 2-retaining surviving), SYT/SSX (synaptotagmin I/synovial sarcoma, X fusion protein), TEL/AML1 (translocation Ets-family leukemia/acute myeloid leukemia 1), TGFβRII (transforming growth factor β receptor 2), TPI (triosephosphate isomerase), TRAG-3 (taxol resistant associated protein 3), TRG (testin-related gene), TRP-1 (tyrosinase related protein 1, or gp75), TRP-2 (tyrosinase related protein 2), TRP-2/INT2 (TRP-2/intron 2), TRP-2/6b (TRP-2/novel exon 6b), WT1 (Wilms' tumor gene). Further tumour associated antigens may be selected from those listed on the world wide web at cancerimmunity.org/peptidedatabase/mutation.htm.

In another embodiment, the antibody may be selected from one which binds to the target antigen CD20. Such antibodies are described in International Patent Application WO 06/130458, which is hereby incorporated by reference. Details of the variable regions of a panel of anti-CD20 antibodies are given in Table 1 of WO 06/130458.

In accordance with an embodiment of the present invention, the antibody that binds to CD20 may comprise the heavy and light chain variable regions of the anti-CD20 antibody 1.5.3 as disclosed in Table 1 of International Patent Application WO 06/130458, which is hereby incorporated by reference. The nucleic acid and protein sequences of the heavy and light chain variable regions of antibody 1.5.3 are given as SEQ ID NOS: 25 to 28 respectively. The anti-CD20 antibody may further comprise an Fc polypeptide variant as provided herein.

Clinical indications in which an Fc polypeptide variant may be used are those in which the polypeptide provides therapeutic benefit.

Such clinical conditions may include cancer, respiratory conditions, inflammation, cardiovascular diseases, gastrointestinal diseases and diseases of the central nervous system.

In accordance with an embodiment of the present invention an Fc polypeptide variant may be given to an individual, preferably by administration in a ‘prophylactically effective amount’ or a ‘therapeutically effective amount’ (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to an embodiment of the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be any suitable route, but most likely injection, especially intravenous injection.

For intravenous, cutaneous or subcutaneous injection, or injection at the site of affliction, the active ingredient will be in the form of a parenterally acceptable aqueous solution which is pyrogen-free (e.g., substantially free of endotoxins and/or related pyrogenic substances) and has suitable pH, isotonicity and stability. Endotoxins include toxins that are confined inside a microorganism and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, it is advantageous to remove even trace amounts of endotoxin. In a specific embodiment, endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

The invention provides methods for preventing, treating, or ameliorating one or more symptoms associated with cancer, said method comprising: (a) administering to a subject in need thereof a dose of a prophylactically or therapeutically effective amount of a composition comprising one or more Fc polypeptide variants and (b) administering one or more subsequent doses of said Fc polypeptide variants, to maintain a plasma concentration of the Fc polypeptide variant at a desirable level (e.g., about 0.1 to about 100 μg/ml), which continuously binds to an antigen. In a specific embodiment, the plasma concentration of the Fc polypeptide variant is maintained at 10 μg/ml, 15 μg/ml, 20 μg/ml, 25 μg/ml, 30 μg/ml, 35 μg/ml, 40 μg/ml, 45 μg/ml or 50 μg/ml. In a specific embodiment, said effective amount of Fc polypeptide variant to be administered is between at least 1 mg/kg and 8 mg/kg per dose. In another specific embodiment, said effective amount of Fc polypeptide variant to be administered is between at least 4 mg/kg and 8 mg/kg per dose. In yet another specific embodiment, said effective amount of Fc polypeptide variant to be administered is between 50 mg and 250 mg per dose. In still another specific embodiment, said effective amount of Fc polypeptide variant to be administered is between 100 mg and 200 mg per dose.

The present invention also encompasses protocols for preventing, treating, or ameliorating one or more symptoms associated with cancer which an Fc polypeptide variant is used in combination with a therapy (e.g., prophylactic or therapeutic agent) other than an Fc polypeptide variant. The invention is based, in part, on the recognition that the Fc polypeptide variants of the invention potentiate and synergize with, enhance the effectiveness of, improve the tolerance of, and/or reduce the side effects caused by, other cancer therapies, including current standard and experimental chemotherapies. The combination therapies of the invention have additive potency, an additive therapeutic effect or a synergistic effect. The combination therapies of the invention enable lower dosages of the therapy (e.g., prophylactic or therapeutic agents) utilized in conjunction with Fc variants for preventing, treating, or ameliorating one or more symptoms associated with a disease, disorder, or infection and/or less frequent administration of such prophylactic or therapeutic agents to a subject with a disease disorder, or infection to improve the quality of life of said subject and/or to achieve a prophylactic or therapeutic effect. Further, the combination therapies of the invention reduce or avoid unwanted or adverse side effects associated with the administration of current single agent therapies and/or existing combination therapies, which in turn improves patient compliance with the treatment protocol. Numerous molecules which can be utilized in combination with the Fc polypeptide variants of the invention are well known in the art. See for example, PCT publications WO 02/070007; WO 03/075957 and U.S. Patent Publication 2005/064514.

Further aspects and embodiments of the present invention will be apparent to those skilled in the art in the light of the present disclosure, including the following experimental exemplification.

All documents mentioned anywhere in this specification are incorporated by reference. In addition, the U.S. provisional patent application 60/895,695 filed Mar. 19, 2007 is incorporated by reference its entirety for all purposes.

### EXPERIMENTAL EXAMPLES

### Example 1

### Selection for Improved Affinity

### Library Construction

Initially, the human Fcγ1 (hFcγ1) heavy chain (CH2:CH3—residues 223-447 by Kabat Eu numbering) was converted to ribosome display format, in either a single domain or as a sequentially displayed dimeric construct (single chain Fc—scFc), whereby two Fc domains are separated by a 30 amino acid linker sequence [(Gly4Ser1)8]. These templates were subsequently used for library creation. To the DNA encoding the hFcγ1 domain, a T7 promoter was added at the 5′-end for efficient transcription to mRNA and sequences for a prokaryotic ribosome binding site such that it was appropriately positioned in the resulting mRNA. Sequences containing 5′ & 3′ stem loops were also added for mRNA stability. At the 3′ end, of the hFcγ1, the stop codon was removed and a sequence encoding a portion of pIII protein from filamentous phage was added to act as a spacer, allowing folding of the hFcγ1 away from the ribosomal tunnel (Hanes et al. (2000) Methods in Enzymology 328: 404).

Where one hFcγ1 domain is displayed per ribosome; two separately displayed hFcγ1 domains come together in solution forming disulphide bridges, at the two cysteine residues in the lower hinge (residues 226 & 229), creating a heterodimeric complex able to bind hFcγ1 receptors (e.g. CD16—hFcγRIIIa). Where an scFc molecule is displayed this binds hFcγ1 receptors without the need for further dimerisation.

Large ribosome display libraries were created by error prone mutagenesis for the entire length (or portions thereof) of the hFcγ1, where PCR reactions were performed with non-proof reading Taq polymerase. The error rate employed created, on average, 4.6 mutations per 1,000 by after a standard PCR reaction, as described in protocol supplied by kit-Diversify™ by Clonetech. In the scFc display molecule only one Fc domain was mutated.

### Selections for Affinity

Affinity based selections were performed where by, following incubation with the library, biotinylated hFcγRIIIa or biotinylated C1q were allowed to bind streptavidin coated paramagnetic beads (Dynal M280). Therefore, the bound tertiary complexes (mRNA-ribosome—hFcγ1) were recovered by magnetic separation whilst unbound complexes were washed away. The mRNA encoding the bound hFcγ1 were then rescued by RT-PCR as described in Hanes et al supra and the selection process repeated with decreasing concentration (500 nM-50 nM over 5 rounds for FcγRIIIa or 100 nM to 100 pM over 3 rounds for C1q) of biotinylated hFc1RIIIa or biotinylated C1q present during the selection. In addition, hybrid selections were performed whereby the hFcγ1 libraries were sequentially selected against multiple antigens (e.g. hFcγRIIIa==>mouse FcγRIIIa==>Protein A), again with increased stringency in selection as the rounds of selection progress. Another embodiment of selections was where selections were performed by alternating the addition of either V158 or F158 allotype of hFcγRIIIa with decreasing antigen concentration along the selection pathway (performed to remove discrimination between hFcγRIIIa V158 and F158 allotypes).

The PCR products from the selections were directionally cloned into pCANTAB6, pUC119FLAG (minus His) or pUC119Bio (minus His). In brief, the PCR products were double digested with the restriction endonucleases Nco 1 and Not 1 (New England Biolabs), digesting at the 5′ and 3′ ends respectively of the DNA encoding hFcγ1. Alternatively, for the scFc construct, digesting at the 5′ end of the DNA encoding the first Fc domain and the 3′ end of the DNA encoding the second Fc domain. The digested DNA was purified by agarose gel electrophoresis to resolve the digested fragments, excision of the fragment encoding the hFcγ1 and purification of the DNA from the agarose slice. The fragment was then ligated into expression vectors (pCANTAB6, pUC119FLAG (minus His) or pUC119Bio (minus His)) opened at their Nco1-Not1 sites and used to transform chemically competent Top10 E. coli cells (Invitrogen 44-0301). Individual colonies were picked for sequencing and preparation for expression.

### Example 2

### Expression of Fc Polypeptide Variants in E. coli

Chemically competent E. coli TOP10 cells (Invitrogen C4040-03) were transformed with Fc polypeptide variants in pUC119Flag using standard practice and plated on agar plates containing nutrients and antibiotics selective for the vector. A single colony was used to inoculate a 10 ml 2TY (containing Amp) starter culture and grown at 37° C. for approximately 8 hours, shaking at 250-300 rpm. 2 ml of this culture was transferred to 400 ml Terrific Broth (containing Ampicillin) and grown overnight (16 hours minimum) at 30° C., shaking at 300 rpm.

Post-growth, the E. coli were harvested by centrifugation. The Fc polypeptide variant was released from E. coli by resuspension of the pellet in buffer (200 mM TrisHCl pH7.4, 0.5 mM EDTA, 0.5M sucrose) containing Lysonase™ (Novagen). The E. Coli suspension was clarified by centrifugation, the supernatant loaded on a Ceramic Protein A (BioSepra) column of appropriate size and washed with 50 mM Tris-HCl pH 8.0, 250 mM NaCl. Bound Fc polypeptide variant was eluted from the column using 0.1 M Sodium Citrate (pH 3.0) and neutralised by the addition of Tris-HCl (pH 9.0). The eluted material was buffer exchanged into PBS using Nap5 columns (Amersham, #17-0853-01) and the concentration of Fc polypeptide variant was determined colorimetrically using the BCA protein quantitation kit (Pierce #23227) using a bacterially sourced scFv as reference standard. The purified Fc polypeptide variants were analyzed for integrity using reducing and non-reducing SDS-PAGE.

### Example 3

### Expression of IgG

Clones were converted from Fc to IgG format by sub-cloning the Fc domains into a vector capable of expressing whole antibody heavy chain. The Fc domains were cloned into a vector (pEU1.2) containing the VH domain (SEQ ID NO: 26) of the anti-CD20 antibody 1.5.3 (as described in International Patent Application WO 06/130458), the human heavy chain constant domain 1 (CH1) and regulatory elements necessary to express whole IgG heavy chain in mammalian cells. The human light chain, used for all variants, was expressed from a vector (pEU3.4) containing the VL domain (SEQ ID NO: 28) of the anti-CD20 antibody 1.5.3 (as described in International Patent Application WO 06/130458), the human light chain (kappa) constant domain and regulatory elements necessary to express whole IgG light chain in mammalian cells. Vectors for the expression of heavy chains and light chains were originally described in Persic et al., (1997, Gene 187(1): 1-8). The vectors described here have been engineered by the introduction of an OriP element, and the introduction of a restriction site to permit the replacement of the wild-type Fc with Fc variants. To obtain IgG, EBNA-HEK293 mammalian cells were transfected with the heavy and light chain expressing vectors. IgGs expressed from the transfected cells were secreted into the medium. Media was harvested at intervals and pooled then filtered prior to purification. The IgG was purified using Protein A chromatography. Culture supernatants are loaded on a Ceramic Protein A (BioSepra) column of appropriate size and washed with 50 mM Tris-HCl pH 8.0, 250 mM NaCl. Bound IgG was eluted from the column using 0.1 M Sodium Citrate (pH 3.0) and neutralised by the addition of Tris-HCl (pH 9.0). The eluted material was buffer exchanged into PBS using Nap10 columns (Amersham, #17-0854-02) and the concentration of IgG was determined spectrophotometrically using an extinction coefficient based on the amino acid sequence of the IgG (Mach et al., (1992) Anal. Biochem. 200(1): 20-6, 74-80). The purified IgG were analysed for aggregation or degradation using SEC-HPLC and by SDS-PAGE.

### Example 4

### Production of Recombinant Receptors

The following extracellular domains of Fcγ receptors were cloned and expressed in pDEST12.2 ORIP vector Flag10His: human FcγRIIaH/R131, human FcγRIIIaF/V158, human FcγRIIb, mouse FcγRII/RIII/RIV, cynomologous FcγRIII, FcRn. The receptor genes were amplified by PCR from a cDNA clone. Initially, they were cloned into pENRTY-D-TOPO vector (Invitrogen, K2400-20) and then transferred into pDEST12.2 ORIP vector for expression in HEK-EBNA cells.

The cells were transfected with the appropriate expression vector using PEI (1:500) in DMEM containing 2% FBS, penicillin and streptomycin. After 24 hours the media was changed to CD-CHO (Invitrogen). The supernatant was harvested at 72, 120 and 192 hours, replacing the media removed with fresh media at the first two harvests. At the end of the harvesting process the supernatants were pooled together, concentrated and used as a crude extract for the purification process.

The entire purification process was performed using an ÄKTA| Explorer. Crude extract was passed over a 5 ml HisTrap|HP column (Amersham Biosciences—17-5248-02) at 5 ml/min and the column flow-through was collected. The column was initially washed with 2×PBS until the absorbance reading at 280 nm returned to baseline. The column was then washed with 10 column volumes of 16 mM imidazole followed by 10 column volumes of 50 mM imidazole. The bound protein was eluted with 20 column volumes of an imidazole gradient from 40 to 400 mM. During the wash and elution steps, 1 ml fractions were collected. Those fractions collected between 70-100% imidazole gradient were retained for analysis and further processing.

The HisTrap| elution fractions (1 ml) were analyzed on Novex 4-12% Bis-Tris gel (NP0323BOX) and the fractions containing the protein of interest were pooled (20 ml total) and the sample was concentrated to 1 ml using an Amicon Ultra-15 30,000 MWCO filter (Millipore—UFC903025). The protein sample was loaded onto Superdex S75 HR 10/30 Gel Filtration Column. The column was run at 0.5 ml/min using 2×PBS. Eluate from the column was monitored for absorbance at 280 nm and consecutive 1 ml fractions collected. The peaks were pooled and analyzed on Novex 4-12% Bis-Tris gel.

The fractions containing the protein of interest were pooled, concentrated and run on 4-12% Bis-Tris. The protein concentration was determined by absorbance at 280 nm and protein purity was analyzed by SDS-PAGE and the protein mass was confirmed by MALDI-TOF-MS.

### Example 5

### Screening of Single Fc Polypeptide Variants in Primary RIA

Fc polypeptide variants were screened for binding to FcγRIIIa using a RIA (radio immunoassay) as described in Jermutus et al. (2001, PNAS 98(1): 75-80). The Fc region from the plasmid for each variant was PCR amplified to produce a linear DNA template. This template was purified and a T7 promoter sequence attached at the 5′ end to enable in vitro transcription. The resulting mRNA was purified using G25 Sephadex™ columns. For each variant, in vitro translations in the presence of 35S-labelled methionine were set up at 37° C. for 40 min. The translations were stopped using PBS with 0.05% Tween 20. The translation mixture was added to a plate coated with 100 nM hFcγRIIIa and incubated for 2 hours at room temperature. The plates were washed three times in PBS with 0.05% Tween 20 and three times in PBS. The bound protein was eluted with 0.1M triethylamine and the radiolabel quantified by liquid scintillation counting. A measure of the variants' improved binding was calculated as the fold improvement in binding against the average binding for wild-type Fc. The more improved the binding of the variant the higher the signal obtained. Results from preliminary RIA screen for Fcγ1 error-prone library post FcγRIIIa selections (100 pM), normalised to Fc-wt on each plate are shown in Table 1 below:

TABLE 1

| | RIA for | |
|---|---|---|
| Clone | RIIIa (V158) | Amino acid changes^{a} |
| wild-type | 1.0 | |
| (normalised) | | |
| 110B09 | 4.4 | N276D, R292Q, V305A, I377V, |
| | | T394A, V412A, K439E |
| 110H08 | 2.7 | P244L, K246E, D399G, K409R |
| 115A02 | 1.4 | S304G, K320R, S324T, K326E, |
| | | M358T |
| 121F02 | 1.7 | F243S, P247L, D265V, V266A, |
| | | S383N, T411I |
| 125B01 | 1.3 | H224N, F243L, T393A, H433P |
| 125E05 | 4.7 | V240A, S267G, G341E, E356G |
| 125H10 | 1.7 | M252T, P291L, P352A, R355W, |
| | | N390D, S408G, S426F, A431S |
| 126F07 | 1.9 | P228L, T289A, L365Q, N389S, |
| | | S440G |
| 126G05 | 1.8 | F241L, V273A, K340Q, L441F |
| 130A02 | 1.4 | F241L, T299A, I332T, M428T |
| 135A09 | 3.4 | E269K, Y300H, Q342R, V422I, |
| | | G446A |
| 135C09 | 1.7 | T225A, R301C, S304G, D312N, |
| | | N315D, L351S, N421S |
| 135D10 | 1.4 | S254T, L306I, K326R, Q362L |
| 135E07 | 1.7 | H224Y, P230S, V323A, E333D, |
| | | K338R, S364C |
| 136D04a | 2.3 | T335I, K414M, P445R |
| 136D04b | 2.3 | T335I, K414M, |
| 136G11 | 2.3 | P247A, E258K, D280N, K288R, |
| | | N297D, T299A, K322E, Q342R, |
| | | S354A, L365P |
| 136H08 | 2.5 | H268N, V279M, A339T, N361D, |
| | | S426P |

^{a}wherein the numbering of the residues is that of the EU index as in Kabat

### Example 6

### AlphaScreen™ Method

The Fcγ1 variants were evaluated by assaying for their inhibition of the interaction between the IgG1 molecule and the V158 or F158 allotype of the FcγRIIIa receptor or H131 or R131 allotype of the FcγRIIa. In this system, the human FcγR was captured and displayed on the surface of the nickel-chelate acceptor beads (Alphascreen Histidine Nickel Chelate detection kit, Perkin Elmer). Biotinylated human IgG1 was captured and displayed on the surface of Streptavidin donor beads (Perkin Elmer); the biotinylated IgG was presented in one of two formats: either as wild type IgG1 (and thus glycosylated) or as the TA299 IgG1 variant, which is aglycosylated due to this mutation.

Variant Fcγ1 domains were investigated in two formats: (1) as bacterially expressed Fcγ1 and (2) as IgG1. This avidity assay provides apparent binding affinities as opposed to equilibrium dissociation constants; for this reason wild-type Fcγ1-FLAG or IgG1 (as appropriate) was investigated in parallel in all assays for direct comparison.

The experimental conditions for the FcγRIIIa V158 assay were as follows: FcγRIIIa V158 (final well concentration 20 nM) was incubated for 30 minutes with Nickel chelate acceptor beads (final well concentration 20 μg/ml). The variant under test was added and incubated for 30 mins. The biotinylated IgG1 and Streptavidin Alphascreen beads were added to the plate. For aglycosyl IgG1, the final well concentration was 10 nM, for glycosylated IgG1, the final well concentration was 1 nM. Alphascreen streptavidin beads were subsequently added to a final well concentration of 20 μg/ml, as per the manufacturer's instructions. The plate was then incubated for a further 60 minutes and then read on the Fusion-α. The data were plotted as log10[inhibitor] against counts using the GraphPad Prism® graphics package.

All incubations with alphascreen beads were performed under green filtered light conditions, as per the manufacturer's instructions. All dilutions of alphascreen beads were performed with 1× alphascreen buffer: 25 mM HEPES, 100 mM NaCl and 0.1% Bovine Serum Albumin (γ-globulin-free, Fraction V, Sigma) pH 7.4.

Analysis of wild type IgG was performed in parallel for all Fc-variant IgG alphascreen inhibition experiments and results are shown in Tables 2, 3, 4 and 5 below.

TABLE 2
GraphPad Prism ® was used to calculate a non-linear
regression for these data and to plot a sigmoidal dose
response curve (variable slope). Where complete curves could
not be generated from the data, the incomplete curves were
assessed qualitatively and a scoring system used to classify
the results i.e. <1 and <<1.
FcγRIIIa V158

| | Mean | | |
|---|---|---|---|
| | IC₅₀ wt | | |
| Clone | I̅C̅₅̅₀̅ c̅l̅o̅n̅e̅ | SEM | n |
| 125B01 | 1.16 | 0.12 | 7 |
| 110H08 | 1.36 | 0.11 | 3 |
| 115A02 | 1.05 | 0.14 | 5 |
| 126F07 | 1.03 | 0.21 | 4 |
| 136D04a | 0.83 | 0.03 | 3 |
| 125H10 | <1 | | 6 |
| 135A09 | <1 | | 6 |
| 136G11 | <<1 | | 4 |
| 130A02 | <<1 | | 8 |

Table 3, 4 and 5: GraphPad Prism® was used to calculate a non-linear regression for these data; however complete curves could not be generated from the data obtained for the F158 inhibition experiments. Therefore, the (incomplete) curves were assessed qualitatively and a scoring system used to classify the results, as follows:

+++ significantly enhanced affinity compared to wild-type
++ enhanced affinity compared to wild-type
+ moderate increase in affinity compared to wild-type
≈ w/t approx equal when compared to wild-type
− moderately below wild-type
−− reduced affinity compared to wild-type
−−− significantly reduced affinity compared to wild type

TABLE 3
FcgRIIIa F158

| Clone | Expt 1 | Expt 2 | Expt 3 | Expt 4 |
|---|---|---|---|---|
| 125B01 | + | − | + | + |
| 110H08 | + | + | + | + |
| 115A02 | n/d | n/d | − | − |
| 126F07 | n/d | n/d | − | − |
| 136D04a | ≈w/t | ≈w/t | − | − |
| 125H10 | ≈w/t | ≈w/t | − | − |
| 135A09 | n/d | n/d | −−− | −−− |
| 136G11 | n/d | n/d | −−− | −−− |
| 130A02 | n/d | n/d | −−− | −−− |

TABLE 4
FcγRIIa-H131

| Clone | Expt 1 | Expt 2 |
|---|---|---|
| 110H08 | + | ++ |
| 126F07 | − | −− |
| 115A02 | −−− | −−− |
| 125B01 | −−− | −−− |
| 125E05 | −−− | n/d |
| 125H10 | −−− | n/d |
| 126G05 | −−− | n/d |
| 121D06 | − | n/d |
| 135C09 | −−− | n/d |
| 135D10 | −− | n/d |
| 136H08 | ≈w/t | ≈w/t |
| 130A02 | −−− | −−− |
| 135A09 | −−− | n/d |
| 135E07 | −−− | n/d |
| 130A02 | −−− | n/d |

TABLE 5
FcγRIIa-R131

| Clone | Expt 1 | Expt 2 | Expt 3 | Expt 4 |
|---|---|---|---|---|
| 110H08 | − | + | + | + |
| 125H10 | −−− | n/d | n/d | n/d |
| 115A02 | − | − | − | − |
| 125B01 | −− | −−− | −−− | n/d |
| 136H08 | ++ | ++ | n/d | n/d |
| 125D02 | −−− | n/d | n/d | n/d |
| 121B11 | −−− | n/d | n/d | n/d |
| 115E10 | −−− | n/d | n/d | n/d |
| 121F9 | −−− | n/d | n/d | n/d |
| 121F02 | −−− | n/d | n/d | n/d |
| 121D06 | −−− | n/d | n/d | n/d |
| 136D04b | + | n/d | n/d | n/d |
| 136D04a | − | − | n/d | n/d |
| 126F07 | −−− | n/d | n/d | n/d |

### Example 7

### ELISA Fcγ Receptors

Variants as IgG1 were assessed for binding to human FcγRIIb and FcγRIIIa, murine FcγRII and FcγRIII and cyno FcγRIII using a three stage ELISA performed in a flat-bottomed microtitre plate. The Fcγ receptor including V158 or F158 for FcγRIIIa or FcγRIIb was coated onto the microtitre plate at a concentration of 100 nM in PBS, and incubated for 2 h at 37° C. or overnight at 4° C. The plate was washed three times with 0.05% Tween-20 in PBS for this and for subsequent washes. The wild-type and variant IgG1 antibodies were each added in 0.05% Tween-20 in PBS titrated 2-fold from 100 nM, and incubated at 37° C. for 2 h. Following incubation (2 h, 37° C.) and three washes, an anti-human Fab′2 specific Fab′2 antibody conjugated to horseradish peroxidase (Jackson Labs) was added to each well of the plate (1 in 5000). After incubation (2 h, 37° C.) and three washes, the wells were developed with TMB (100 μl), quenching the reaction by the addition of 20 μl of 4M H₂SO₄. The absorbances were measured at 450 nm using a Wallac 1420 Victor plate reader.

From the panel tested, a number of variants of human IgG1 were identified that bound to the receptors tested and these are shown in Tables 6 and 7 and summarized for all receptors tested in Table 8 below:

TABLE 6
human FcγRIIIa V158

| | ½ Amax (+) | | |
|---|---|---|---|
| Clone ID | EC50 [a] | Amax [b] | [a] × [b] |
| wild-type | 1 | 1 | 1 |
| 110B09 | 0.5 ± .5 | 0.9 ± .6 | 0.5 |
| 110H08 | 2.0 ± .1 | 1.0 ± 0 | 2.0 |
| 115A02 | 0.8 ± .5 | 1.0 ± .4 | 0.8 |
| 125B01 | 1.9 ± .8 | 1.0 ± .5 | 1.9 |
| 125H10 | 1.3 ± .3 | 1.9 ± .2 | 2.5 |
| 126F07 | 1.3 ± .4 | 1.1 ± .2 | 1.4 |
| 126G05 | 0.2 ± .1 | 0.6 ± .1 | 0.1 |
| 130A02 | 5.0 ± 1.8 | 1.5 ± .4 | 7.5 |
| 135A09 | 7.2 ± .3 | 1.4 ± .3 | 10.1 |
| 135D10 | 0.5 ± .1 | 0.8 ± .1 | 0.4 |
| 135E07 | 0.4 ± .1 | 0.9 ± .1 | 0.4 |
| 136D04a | 0.9 ± .2 | 1.4 ± .8 | 1.3 |
| 13GD04b | 0.9 ± .2 | 1.2 ± .3 | 1.1 |
| 136G11 | 1.4 ± .9 | 0.6 ± .1 | 0.8 |
| 136H08 | 0.6 ± .1 | 0.8 ± .1 | 0.5 |

TABLE 7
human FcγRIIb

| | ½ Amax (+) | | |
|---|---|---|---|
| Clone ID | EC50 [a] | Amax [b] | [a] × [b] |
| wild-type | 1 | 1 | 1 |
| 110H08 | 1.9 ± .5 | 1.5 ± .3 | 2.9 |
| 115A02 | 0.3 ± .2 | 0.7 ± .4 | 0.2 |
| 125B01 | 0.7 ± .1 | 0.9 ± 0 | 0.6 |
| 125H10 | 0.4 ± 0 | 0.8 ± .2 | 0.3 |
| 126F07 | 1.8 ± .3 | 1.1 ± 0 | 2.0 |
| 135A09 | 1.1 ± .5 | 1.3 ± .3 | 1.4 |
| 136D04a | 1.4 ± .1 | 0.9 ± .1 | 1.3 |
| 136G11 | 1.1 ± .3 | 1.2 ± .1 | 1.3 |

TABLE 8

| | FcγRIIIa | FcγRIIIa | | | |
|---|---|---|---|---|---|
| Clone ID | V158 | F158 | mFcγRIII | cFcγRIII | FcγRIIb |
| wild-type | 1 | 1 | 1 | 1 | 1 |
| 110H08 | 2 | 4.8 | 1.4 | 8.1 | 2.9 |
| 115A02 | 0.8 | 13.3 | 3.5 | 1.1 | 0.2 |
| 125B01 | 1.9 | 8.4 | 0 | 0.9 | 0.6 |
| 125H10 | 2.5 | 77.5 | 0* | 0.7 | 0.3 |
| 126F07 | 1.4 | 52.3* | 0.9 | | 2.0 |
| 130A02 | 7.5 | 289 | | | |
| 135A09 | 10.1 | 359 | 0 | | 1.4 |
| 136D04a | 1.3 | 3.1 | 0.4 | 1.7 | 1.3 |
| 136D04b | 1.1 | 0.8* | | | |
| 136G11 | 0.8 | 69.0* | 0 | | 1.3 |
| 136H08 | 0.5 | 0.6* | | | |

| | FcγRIIa | FcγRIIa | | | |
|---|---|---|---|---|---|
| Clone ID | H131 | R131 | mFcγRII | FcγRI | C1q |
| wild-type | 1 | 1 | 1 | 1 | 1 |
| 110H08 | 9.5 | 5.5 | 0.4 | 1.1 | 1 |
| 115A02 | 0.3 | 0.1 | 4.8 | 0.8 | 4.8 |
| 125B01 | 1.3 | 0.4 | <0.3 | 0.5 | 0 |
| 125H10 | 2.3 | 0.6 | <0.2 | 0.3 | 5.9 |
| 126F07 | | | | | |
| 130A02 | | | | | |
| 135A09 | | | | | |
| 136D04a | 1.3 | 0.6 | 0.4 | 0.5 | 2.9 |
| 136D04b | | | | | |
| 136G11 | | | | | |
| 136H08 | | | | | |

### Example 8

### ADCC Assays

The ability of Fc variants to trigger FcγRIIIa Mediated effector functions was determined by cell based ADCC assay. The variable region of an IgG antibody binds target antigen on cells and the Fc region of this antibody is then recognised by FcγRIIIa on Natural Killer (NK) cells. Signals delivered to the NK cells via FcγRIIIa subsequently prompt the NK cells to mediate lysis of the antibody bound target cells.

The process of ADCC can be reconstituted in vitro using cultured target cells, antibody in solution and NK effector cells isolated from human blood. Lysis of the target cells is measured, in this methodology, using the aCella-Tox kit (Cell Technologies), which links release of the enzyme Glyceraldehyde-3-phosphate Dehydrogenase (GAPDH) from lysed target cells to ATP release and subsequent luminescence via luciferase. Luminescence from each sample, measured using a luminometer, is then proportional to the number of lysed cells in a given sample.

All Fcγ1 domains were investigated as IgG1 antibodies directed against the CD20 antigen, and expressed in HEK-EBNA cells. VH and VL domains (SEQ ID NOS: 26 & 28, respectively) of antibody 1.5.3 as described in International Patent Application WO 06/130458, were used in the preparation of the IgG1 antibodies. These IgG1 antibodies were evaluated by their ability to mediate increased ADCC relative to an IgG of the same format, but carrying a wild type Fcγ1 domain. Target cells were all CD20 expressing human B cell lines each established from patients presenting with a different hematological malignancy. Specifically, the cell lines used were Daudi B cells, established from a Burkitt's lymphoma patient, EHEB B cells, established from a B chronic lymphocytic leukemia patient, and Karpas-422, established from a patient with diffuse large B cell lymphoma. Effector cells in all experiments were human NK cells isolated from human blood buffy coats. Buffy coats were obtained from the Blood Transfusion Service at Addenbrookes Hospital, Cambridge, UK. Peripheral blood mononuclear cells (PBMCs) were recovered from blood by layering over Histopaque (Sigma) followed by centrifugation at 400 g for 40 min, with no brake. NK cells were purified from PBMCs by magnetic cell separation using the Human NK cell isolation kit and LS columns (Miltenyi Biotec). This is a negative selection, which provides untouched human NK cells at greater than 98% purity.

Target B cells were added to a 96 well round bottom tissue culture plate in RPMI 1640 Glutamax I (GIBCO) supplemented with 10% low IgG FBS (GIBCO) and 1% penicillin/streptomycin (GIBCO); referred to subsequently as media. Cells were added in a volume of 50 μl at a density of 5000 cells per well. Each Fcγ1 variant IgG was diluted to a concentration of 24 nM in media and then used to make a 7 step 1 in 3 serial dilution. 50 μl of each dilution was added to a separate well of target cells. Finally NK effector cells were added to each well in a volume of 100 μl in order to give the indicated effector to target ratio (E:T).

All reaction wells were set up in triplicate. Final well volumes were 200 μl and all empty wells were filled with PBS. Final concentrations of antibody ranged from 6 nM to 0.003 nM. Control wells were included as follows: 3× media alone; 6×5000 target B cells alone; 3×5000 target B cells together with NK effector cells but with no antibody; 3×NK effector cells alone.

Once set up plates were incubated for 2 hours at 37° C. in 5% CO₂. 15 minutes prior to the end of the reaction, 3 of the wells containing 5000 target B cells alone were lysed completely by addition of 10 μl of lysis agent (Cell Technologies); these wells provide a value for 100% lysis of the target cells. At the end of the incubation period reaction plates were centrifuged at 400 g for 4 minutes in order to pellet cells and dead cell debris. 100 μl of supernatant was removed from the reaction plate to the equivalent position in a 96 well optiplate (Perkin Elmer). Supernatants were then assayed for GAPDH levels using the aCella-Tox kit (Cell Technologies). After addition of assay reagents as per manufacturer's instructions the optiplates were sealed and left in the dark for 10 mins, they were then read using a Berthold LB940 Luminometer. In order to calculate ADCC at each antibody concentration all triplicate luminescence values were averaged and corrected for media background. The following formula was then employed:

$$\% ADCC = \frac{\begin{matrix}Sample \\ luminescence\end{matrix} - \begin{matrix}Target only \\ luminescence\end{matrix} - \begin{matrix}NK cells only \\ luminescence\end{matrix}}{100 \% Lysis}$$

Standard deviations were propagated throughout all calculations in order to allow calculation of standard errors. The data were plotted as log10[Antibody] against % ADCC using the Prism graphics package.

Results are for each 158 FcγRIIIa allotype (158FF, VV or VF) and are shown in Tables 9 to 11 below. Tables 9a, 10a and 11a show the fold change of anti-CD20 antibody (1.5.3) variant from 50% of the standard value set using rituximab. Tables 9b, 10b and 11b show the fold change of antibody from the 50%*Max value. This value takes into account the concentration of antibody at which ADCC reached 50% of the standard value set using rituximab and the maximum value from the curve generated using the GraphPad Prism® graphics package. Fold change is calculated by dividing the value for wild-type Fc polypeptide by that of the Fc polypeptide variant in question.

TABLE 9a
Fold change of antibody variant from 50% of the
standard value set using rituximab; Daudi target cells

| Clone | F/F | | V/V | | | V/F | |
|---|---|---|---|---|---|---|---|
| Exp No | 1 | 2 | 1 | 2 | 3 | 1 | 2 |
| WT_0605 | 1.00 | 1.00 | 1.00 | 1.00 | 1.00 | 1.00 | 1.00 |
| 110H08 | 2.90 | 0.53 | 1.04 | 1.11 | 1.03 | 1.70 | |
| 115A02 | | | | | 1.31 | 1.46 | 0.43 |
| 121F02 | | | | | | 0.00 | 0.00 |
| 125B01 | 26.12 | 2.99 | 3.01 | 3.82 | 2.72 | 3.08 | 1.59 |
| 125E05 | | 0.37 | | | | | |
| 125H10 | | | | | | 0.00 | 0.00 |
| 126F07 | | | 0.94 | 1.08 | | | |
| 135A09 | | | | | | 0.00 | 0.00 |
| 135C09 | | | | | 0.00 | | |
| 135D10 | | 0.03 | | | | | |
| 136D04b | | | 1.22 | 1.20 | | | |

TABLE 9b
Fold change of antibody variant from 50%*Max of the
standard value set using rituximab; Daudi target cells

| Clone | F/F | | V/V | | | V/F | |
|---|---|---|---|---|---|---|---|
| Exp No | 1 | 2 | 1 | 2 | 3 | 1 | 2 |
| WT_0605 | 1.00 | 1.00 | 1.00 | 1.00 | 1.00 | 1.00 | 1.00 |
| 110H08 | 0.41 | 0.43 | 0.94 | 1.07 | 1.12 | 2.11 | |
| 115A02 | | | | | 1.32 | 1.47 | 0.49 |
| 121F02 | | | | | | | |
| 125B01 | 6.18 | 5.30 | 3.79 | 4.20 | 3.67 | 4.01 | 2.06 |
| 125E05 | | 0.02 | | | | | |
| 125H10 | | | | | | | |
| 126F07 | | | 1.00 | 0.79 | | | |
| 135A09 | | | | | | | |
| 135C09 | | | | | | | |
| 135D10 | | 0.37 | | | | | |
| 136D04b | | | 1.36 | 1.00 | | | |

TABLE 10a
Fold change of antibody variant from 50% of the
standard value set using rituximab; EHEB target cells

| | | |
|---|---|---|
| Clone | F/F | V/F |

| | | | |
|---|---|---|---|
| Exp No | 1 | 1 | 2 |
| WT | 1 | 1 | 1 |
| 125_B01 | 1.90 | 1.74 | 1.17 |

TABLE 10b
Fold change of antibody variant from 50%*Max of the
standard value set using rituximab; EHEB target cells

| | | |
|---|---|---|
| Clone | F/F | V/F |

| | | | |
|---|---|---|---|
| Exp No | 1 | 1 | 2 |
| WT | 1 | 1 | 1 |
| 125_B01 | 9.5 | 5.23 | 4.00 |

TABLE 11a
Fold change of antibody variant from 50% of the
standard value set using rituximab; Karpas-422 target cells

| | | |
|---|---|---|
| Clone | V/V | V/F |

| | | | |
|---|---|---|---|
| Exp No | 1 | 1 | 2 |
| WT | 1 | 1 | 1 |
| 110H08 | 1.89 | 2.03 | 1.03 |

TABLE 11b
Fold change of antibody variant from 50%*Max of the
standard value set using rituximab; Karpas-422 target cells

| | | |
|---|---|---|
| Clone | V/V | V/F |

| | | | |
|---|---|---|---|
| Exp No | 1 | 1 | 2 |
| WT | 1 | 1 | 1 |
| 110H08 | 6.04 | 10.01 | 3.76 |

### Example 9

### RIA Primary Screen

### C1q

IgG1 Fc variants arising from round three outputs of Fcγ1 selections against human C1q using ribosome display, were screened for binding using the primary RIA as described in Jermutus et al. (2001, PNAS 98(1): 75-80). In brief, the Fc region from the plasmid for each variant (96 well format) was PCR amplified to produce a linear DNA template. This template was gel purified and reamplified attaching the T7 promoter sequence for T7 directed in vitro transcription and the resulting mRNA purified on G25 Sephadex columns. For each variant, in vitro translations in the presence of 35S-labelled methionine was performed at 37° C. for 40 mins. The translations were stopped with PBS with 0.05% Tween 20. The translation mixture was incubated on a plate coated with 100 nM hC1q for 2 hours at room temperature. Plates were washed three times in PBS with 0.05% Tween 20 and three times in PBS. The remaining radioactivity was eluted with 0.1M triethylamine and quantified by liquid scintillation counting. A measure of the variants' improved binding was calculated as the fold improvement in binding against the average binding for wild-type IgG1 Fc derived from clones A02 and E05 on the same plate. The more improved the binding of the variant the higher the signal obtained.

The results, shown below in Table 12, indicate up to 4-fold greater binding of 35S-Met labelled variants to immobilised C1q, relative to the wild-type IgG1-Fc, given by samples A02 and E05. The mutations are scattered through the CH2 and CH3 regions with several mutations present at the interface between the CH3 domains.

TABLE 12

| Clone | RIA for C1q | Amino acid changes^{a} |
|---|---|---|
| wild type | 1.0 | |
| (normalised) | | |
| 2A11 | 2.4 | C261Y, K290E, L306F, Q311R, |
| | | E333G, Q438L |
| 2C06 | 1.4 | E283G, N315K, E333G, R344Q, |
| | | L365P, S442T |
| 3C11 | 1.2 | Q347R, N361Y, K439R |
| 2F01 | 1.5 | S239P, S254P, S267N, H285R, |
| | | N315S, F372L, A378T, N390D, |
| | | Y391C, F404S, E430K, L432P, |
| | | K447E |
| 2H06 | 1.2 | E269G, Y278H, N325S, K370R |

^{a}wherein the numbering of the residues is that of the EU index as in Kabat

### Example 10

### ELISA

### C1q

Variants from ribosome display selection of Fcγ1 variants for binding to human C1q were converted into intact IgG format as described above in Example 3. C1q binding was then assessed using a four stage ELISA performed in a flat-bottomed microtitre plate. NIP-ovalbumin was made by reacting 220 μM OVA in borate-buffered saline with a 100-fold molar excess of NIP-caproate-O-succinimide for 30 min at room temperature, followed by dialysis against PBS and storage at −20° C. The NIP-OVA was coated onto the microtitre plate at a concentration of 0.11 μM (100 μl) in PBS, and incubated for 2 h at 37° C. The plate was washed three times with 0.05% Tween-20 in PBS for this and for subsequent washes. The IgG antibodies were each added in 0.05% Tween-20 in PBS (100 μl at 30 nM), and incubated at 37° C. for 2 h. Human C1q (Sigma) was added to the first well of the plate at a concentration of 30 nM or 40 nM, and then 2-fold serially diluted in PBS-Tween-20. Following incubation (2 h, 37° C.) and three washes, a sheep anti-human C1q antibody conjugated to horseradish peroxidase was added to each well (30 nM). After incubation (2 h, 37° C.) and three washes, the wells were developed either with peroxidase substrate (100 μl; 48 mM sodium citrate, 96 mM disodium hydrogen phosphate, 3 mM H₂O₂ and 2 mM o-phenylenediamine) or with tetramethylbenzidine, quenching the reaction by the addition of 20 μl of 4M H₂SO₄. The absorbances were measured at 490 nM or 450 nM respectively, using a Wallac 1420 Victor plate reader.

From the panel tested, a number of variants of human IgG1 were identified that bound to the receptor and these are shown in Table 13 below. Variant 2F01 from ribosome display selection of Fcγ1 variants was compared with the wild-type. 2F01 gave a signal around 32 fold greater than the mean signal for the wild-type.

TABLE 13

| | ½ Amax (+) | | |
|---|---|---|---|
| Clone ID | EC50 [a] | Amax [b] | [a] × [b] |
| wild-type | 1 | 1 | 1 |
| 3C11 | 0.8 ± 0 | 1.0 ± .1 | 0.8 |
| 2H06 | 0.5 ± .1 | 0.9 ± .1 | 0.5 |
| 2F01 | 24.4 ± 15.1 | 1.3 ± .2 | 31.7 |
| 2A11 | 0.8 ± .2 | 1.1 ± .2 | 0.9 |
| 2C06 | 0.6 ± .3 | 0.8 ± .2 | 0.5 |

### Example 11

### Oligosaccharide Analysis of Improved Variants

The complex oligosaccharide attached to Asn-297 of IgG-Fc is known to modulate recognition by effector ligands and consequently effector functions. For this reason oligosaccharide analysis was carried out by the National Institute for Bioprocessing Research and Training (NIBRT; Dublin) on wild-type and two variant anti-CD20 IgG1 antibodies, each expressed in HEK293 cells. The anti-CD20 antibodies were constructed with VH and VL domains (amino acid SEQ ID NOS: 26 & 28, respectively) of antibody 1.5.3 as described in International Patent Application WO 06/130458. Briefly, oligosaccharides were released from IgG by enzymatic digestion, derivatized with a fluorescent reagent and analysed by HPLC, relative to known oligosaccharide standards as described in inter alia: Guile et al, (1996) Anal. Biochem., 240: 210; Arnold et al, (2004) J. Immunol., 173: 6831; Royle et al, (2006) Current Protocols in Protein Science, 12.6.1-12.6.45. The oligosaccharide aminobenzamide derivatives were typed by relative mobility on an HPLC system, and can be identified via GlycoBase (on the world wide web at glycobase.ucd.ie/cgi-bin/public/glycobase.cgi), run by NIBRT. The analyses data are summarised in Table 14 below:

TABLE 14
The values shown are percentage values of total peak
intensity from HPLC readouts and are therefore quantitative.

| | wild-type | 110H08 | 125B01 |
|---|---|---|---|
| Gal0 | 46.3 | 27.1 | 14.9 |
| Gal1 | 43.8 | 50.0 | 21.6 |
| Gal2 | 9.9 | 22.9 | 63.5 |
| bGlcNAc | 3.8 | 5.7 | 12.9 |
| afucosylated | 3.8 | 6.8 | 21.8 |
| sialylated | 1.0 | 2.6 | 16.1 |

The results in Table 14 indicate differences in oligosaccharide profiles between the wild-type and variant anti-CD-20 IgG1 antibodies, 110H08 and 125B01. Thus, relative to the wild-type, the variants have increased levels of oligosaccharide chains with galactose (Gal1, Gal2), with sialic acid, with bisecting N-acetylglucosamine, or without fucose residues. Both the increased level of afucosylated oligosaccharide chains seen in variant 125B01 and the increased level of oligosaccharides bearing bisecting N-acetylglucosamine may account for the enhanced recognition by FcγRIIIa and ADCC by NK cells, relative to the wild-type IgG.

### Example 12

### Oligosaccharide Analysis of Anti-CD20 Antibody 243 Mutants

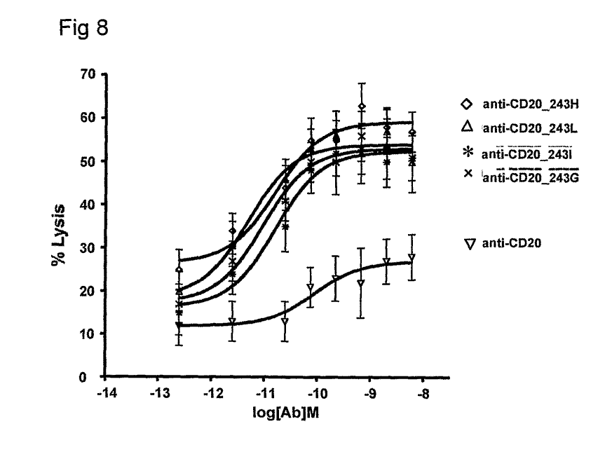

Deconvolution work on the anti-CD20 variant antibody 125B01 (as described in Example 12) revealed the amino acid at position 243 to be key to receptor binding and effector function. Therefore, a number of variant anti-CD20 IgGs with a point mutation at position 243 were generated (F243A, R, N, D, E, H, K, M, S, T, W, Y, V, P, I, G and Q). These variants were transfected into CHO EBNA cells using linear PEI, purified by affinity chromatography on protein A and their efficacy and potency tested in ADCC assays (for ADCC method see Example 8; ADCC results for F243H, L, I and G variants are shown in FIG. 8). All of the variant IgGs showed activity in the ADCC assay with the 243L variant showing the most improved efficacy and potency over wild-type anti-CD20 IgG. Variants 243G, 243H and 243I also showed a marked improvement in efficacy and potency over wild-type anti-CD20 antibody.

Oligosaccharide analysis was then performed in-house for a wild-type anti-CD20 IgG, the anti-CD20 variants L243, G243 and H243 and Rituxan. An aliquot of antibody (100 μg) was dissolved in 0.1M ammonium bicarbonate (28 μL) and deglycosylated by incubating with N-Glycanase (5 units) for 18 hours at 37° C. The oligosaccharides were isolated by microcentrifugation, concentrated to dryness in a vacuum centrifuge and labeled with a Glyko Signal 2-AB labeling kit (Europa Bioproducts Ltd). Post-label clean-up was performed with an Oasis HLB Extraction Cartridge (Waters). The labeled oligosaccharides were concentrated to dryness and dissolved in 70% acetonitrile (100 μL). 10 μL of sample were injected onto a GlycoSep N normal-phase HPLC column (250 mm×4.6 mm) and separated using the method established by Guile et al (supra). Solvent A was acetonitrile and solvent B was 50 mM ammonium formate pH 4.4. The following gradient conditions were used: t=0 min, 35% solvent B; t=70 min, 50% solvent B. The flow rate was 400 μL/min. Labeled oligosaccharides were monitored using a Dionex RF2000 fluorescence detector (excitation λ 330 nm, emission λ 420 nm) and identified by reference to 2-AB glucose homopolymer GU values.

The oligosaccharide profile is shown in Table 15 below. The increased levels of afucosylated oligosaccharides relative to wild-type oligosaccharide chains correlate with the enhanced ADCC results for the anti-CD20 IgG variants L243, G243 and H243 and indicates that a mutation at position 243 to L, G or H results in increased levels of afucosylated oligosaccharides relative to wild-type.

TABLE 15
The values shown are percentage values of total peak
intensity from HPLC readouts and are therefore quantitative.

| | | Anti-CD20 | Variant | Variant | Variant |
|---|---|---|---|---|---|
| | Rituxan | antibody | 234L | 243G | 243H |
| Gal0 | 75.4 | 78.4 | 16.7 | 15.4 | 15.3 |
| Gal1 | 22.3 | 21.6 | 34.5 | 33.3 | 30.8 |
| Gal2 | 1.5 | | 47.1 | 19.9 | 52.9 |
| afucosylated | 7.1 | 10.1 | 34.9 | 35.7 | 36.5 |
| sialylated | | | 19.8 | 21.6 | 25.5 |

1. A method of providing an Fc polypeptide variant with altered recognition of an Fc ligand and/or improved effector function compared with a parent Fc polypeptide, the method comprising:
(a) providing mRNA molecules, each mRNA molecule comprising a nucleotide sequence encoding an Fc polypeptide variant and lacking an in-frame stop codon;
(b) incubating the mRNA molecules under conditions for ribosome translation of the mRNA molecules to produce encoded Fc polypeptide variants, whereby complexes each comprising at least mRNA and encoded Fc polypeptide variant are formed;
(c) bringing the complexes into contact with an Fc ligand that binds the parent Fc polypeptide, and selecting one or more complexes each displaying an Fc polypeptide variant able to bind the Fc ligand under the conditions of the selection;
(d) determining recognition or effector function of selected Fc polypeptide variant or variants, whereby one or more Fc polypeptide variants with improved Fc ligand recognition and/or improved effector function compared with the parent Fc polypeptide are obtained;
(e) retrieving mRNA from a selected complex; and
(f) amplifying and copying the retrieved mRNA into DNA encoding the selected Fc polypeptide variant.

2-3. (canceled)

4. The method of claim 1, wherein the Fc ligand is an Fc receptor or C1q.

5. The method of claim 4, wherein the Fc receptor is selected from FcγRI, FcγRII or FcγRIII families.

6-7. (canceled)

8. The method of claim 1, wherein the altered recognition of an Fc ligand is reduced binding to FcγRIIb.

9-10. (canceled)

11. The method of claim 1, wherein the altered recognition of an Fc ligand is improved binding to FcγRIIIa.

12. The method of claim 1, wherein the effector function is ADCC or CDC.

13-16. (canceled)

17. The method of claim 1, wherein the Fc polypeptide variant comprises a variant human IgG Fc region.

18. The method of claim 17, wherein the IgG Fc region is selected from IgG1, IgG2, IgG3 or IgG4.

19-22. (canceled)

23. The method of claim 1 wherein the DNA encoding the selected Fc polypeptide variant. is provided in an expression system.

24-34. (canceled)

35. An Fc polypeptide variant having a mutation at two or more positions in the amino acid sequence of human wild-type sequence of SEQ ID NO: 1, wherein the residue provided at any one of said positions is selected from the following: 224N/Y, 225A, 228L, 230S, 239P, 240A, 241L, 243S/L/G/H/I, 244L, 246E, 247L/A, 252T, 254T/P, 258K, 261Y, 265V, 266A, 267G/N, 268N, 269K/G, 273A, 276D, 278H, 279M, 280N, 283G, 285R, 288R, 289A, 290E, 291L, 292Q, 297D, 299A, 300H, 301C, 304G, 305A, 306I/F, 311R, 312N, 315D/K/S, 320R, 322E, 323A, 324T, 325S, 326E/R, 332T, 333D/G, 335I, 338R, 339T, 340Q, 341E, 342R, 344Q, 347R, 351S, 352A, 354A, 355W, 356G, 358T, 361D/Y, 362L, 364C, 365Q/P, 370R, 372L, 377V, 378T, 383N, 389S, 390D, 391C, 393A, 394A, 399G, 404S, 408G, 409R, 411I, 412A, 414M, 421S, 422I, 426F/P, 428T, 430K, 431S, 432P, 433P, 438L, 439E/R, 440G, 441F, 442T, 445R, 446A, 447E, wherein the variant has altered recognition of an Fc ligand and/or altered effector function compared with a parent Fc polypeptide, and wherein the numbering of the residues is that of the EU index as in Kabat.

36. (canceled)

37. The Fc polypeptide variant of claim 35 comprising a set of mutations in the human wild-type sequence of SEQ ID NO: 1 selected from the group consisting of the following sets of mutations:
(2) N276D, R292Q, V305A, I377V, T394A, V412A and K439E;
(3) P244L, K246E, D399G and K409R;
(4) S304G, K320R, S324T, K326E and M358T;
(5) F243S, P247L, D265V, V266A, S383N and T411I;
(6) H224N, F243L, T393A and H433P;
(7) V240A, S267G, G341E and E356G;
(8) M252T, P291L, P352A, R355W, N390D, S408G, S426F and A431S;
(9) P228L, T289A, L365Q, N389S and 5440G;
(10) F241L, V273A, K340Q and L441F;
(11) F241L, T299A, I332T and M428T;
(12) E269K, Y300H, Q342R, V422I and G446A;
(13) T225A, R301c, S304G, D312N, N315D, L351S and N421S;
(14) S254T, L306I, K326R and Q362L;
(15) H224Y, P230S, V323A, E333D, K338R and S364C;
(16) T335I, K414M and P445R;
(17) T335I and K414M;
(18) P247A, E258K, D280N, K288R, N297D, T299A, K322E, Q342R, S354A and L365P;
(19) H268N, V279M, A339T, N361D and S426P;
(20) C261Y, K290E, L306F, Q311R, E333G and Q438L;
(21) E283G, N315K, E333G, R344Q, L365P and S442T;
(22) Q347R, N361Y and K439R;
(23) S239P, S254P, S267N, H285R, N315S, F372L, A378T, N390D, Y391C, F404S, E430K, L432P and K447E; and
(24) E269G, Y278H, N325S and K370R,
wherein the numbering of the residues is that of the EU index as in Kabat.

38. (canceled)

39. The Fc polypeptide variant of claim 35, wherein the Fc ligand is an Fcγ receptor or C1q.

40. The Fc polypeptide variant of claim 39, wherein the Fcγ receptor is selected from FcγRI, FcγRII or FcγRIII families.

41-46. (canceled)

47. The Fc polypeptide variant of claim 35, wherein the altered effector function is improved ADCC or CDC.

48-67. (canceled)

68. A method of increasing the percentage of Fc polypeptides comprising a mature core carbohydrate structure which lacks fucose present in a composition to at least 20%, said method comprising:
a. introducing at least one mutation into a nucleic acid encoding the Fc polypeptide, wherein the mutation results in a substitution at amino acid residue 243; and
b. expressing the mutated nucleic acid in an animal cell to produce a glycosylated composition of Fc polypeptides,
wherein the numbering of the residues is that of the EU index as in Kabat.

69-70. (canceled)

71. The method of claim 68, wherein the mutation results in a substitution at position 243 selected from the group consisting of 243L, 243G, 243H and 243I.

72-73. (canceled)

74. The method of claim 68, wherein the percentage of Fc polypeptides comprising a mature core carbohydrate structure which has sialic acid present in the composition is increased to at least 10%.

75-76. (canceled)

77. The method of claim 68, wherein the percentage of Fc polypeptides comprising a mature core carbohydrate structure which has bisecting GlcNAc present in the composition is increased to at least 5%.

78. A method of increasing the percentage of Fc polypeptides comprising a mature core carbohydrate structure which has sialic acid present in a composition to at least 10%, said method comprising:
a. introducing at least one mutation into a nucleic acid encoding the Fc polypeptide, wherein the mutation results in a substitution at amino acid residue 243; and
b. expressing the mutated nucleic acid in an animal cell to produce a glycosylated composition of Fc polypeptides,
wherein the numbering of the residues is that of the EU index as in Kabat.

79-80. (canceled)

81. The method of claim 78, wherein the mutation results in a substitution at position 243 selected from the group consisting of 243L, 243G, 243H and 243I.
